(19) United States
(12) Patent Application Publication
(10) Pub. No.: US 2023/0174993 A1
(43) Pub. Date: Jun. 8, 2023

(54) BIOSYNTHESIS OF MOGROSIDES

(71) Applicant: Ginkgo Bioworks, Inc., Boston, MA (US)

(72) Inventors: Jeffrey Ian Boucher, Allston, MA (US); Justin Michael Gardin, Watertown, MA (US); Scott Marr, Boston, MA (US); Matthew McMahon, Melrose, MA (US); Krishnaben S. Patel, Melrose, MA (US); Jie Zhu, Watertown, MA (US)

(73) Assignee: Ginkgo Bioworks, Inc., Boston, MA (US)

(21) Appl. No.: 17/924,659

(22) PCT Filed: May 13, 2021
(86) PCT No.: PCT/US2021/032251

§ 371 Date: Nov. 10, 2022

Related U.S. Application Data
(60) Provisional application No. 63/024,392, filed on May 13, 2020.

(51) Int. Cl. C12N 15/52; C12N 15/81; C12P 19/56; C12P 33/02

(52) U.S. Cl. CPC C12N 15/52; C12N 15/81; C12P 19/56; C12P 33/02; C12Y 114/15004; C12Y 303/0201; C12Y 303/02009; C12Y 504/99033

(57) ABSTRACT

Described in this application are UDP-glycosyltransferases (UGT) enzymes, host cells expressing the UGTs, and methods of producing mogrol precursors, mogrol, and/or mogrosides using such host cells.

### CROSS REFERENCE TO RELATED APPLICATIONS

This application claims the benefit under 35 U.S.C. § 119(e) of U.S. Provisional Application No. 63/024,392, filed May 13, 2020, entitled “BIOSYNTHESIS OF MOGROSIDES,” the entire disclosure of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on May 13, 2021, is named G091970061WO00-SEQ-FL.txt and is 888,111 bytes in size.

### FIELD OF THE INVENTION

The present disclosure relates to the production of mogrol precursors, mogrol and mogrosides in recombinant cells.

### BACKGROUND

Mogrosides are glycosides of cucurbitane derivatives. Highly sought after as sweeteners and sugar alternatives, mogrosides are naturally synthesized in the fruits of plants, including Siraitia grosvenorii ( S. grosvenorii). Although anti-cancer, anti-oxidative, and anti-inflammatory properties have been ascribed to mogrosides, characterization of the exact enzymes involved in mogroside biosynthesis is limited. Furthermore, mogroside extraction from fruit is labor-intensive and the structural complexity of mogrosides often hinders de novo chemical synthesis.

### SUMMARY

Aspects of the present disclosure provide host cells that comprise a heterologous polynucleotide encoding a UDP-glycosyltransferase (UGT). In some embodiments, the UGT comprises a sequence that is at least 90% identical to any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 94 and 100, wherein the UGT is capable of catalyzing conversion of mogrol to MIA1.

In some embodiments, the UGT comprises a sequence that is at least 90% identical to any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, and 84. In some embodiments, the UGT comprises a sequence that is at least 90% identical to SEQ ID NO: 76. In some embodiments, the UGT comprises a sequence that is at least 90% identical to SEQ ID NO: 66. In some embodiments, the UGT comprises SEQ ID NO: 88.

In some embodiments, the UGT comprises the sequence of any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 94, and 100. In some embodiments, the UGT comprises the sequence of any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, and 84. In some embodiments, the UGT comprises SEQ ID NO: 76.

Further aspects of the present disclosure provide host cells that comprise a heterologous polynucleotide encoding a UDP-glycosyltransferase (UGT), wherein the UGT comprises a sequence that is at least 90% identical to any one of SEQ ID NOs: 103, 74, 79, 102, 62, and 87 and wherein the UGT is capable of catalyzing conversion of mogrol to MIE1.

In some embodiments, the UGT comprises the sequence of any one of SEQ ID NOs: 103, 74, 79, 102, 62, and 87.

Further aspects of the present disclosure provide host cells that comprise a heterologous polynucleotide encoding a UDP-glycosyltransferase (UGT), wherein the UGT comprises a sequence that is at least 90% identical to any one of SEQ ID NOs: 63, 77, 84, and 94, and wherein the UGT is capable of catalyzing conversion of mogrol to MIIE1.

In some embodiments, the UGT comprises the sequence of any one of SEQ ID NOs: 63, 77, 84, and 94.

In some embodiments, the host cell further comprises one or more heterologous polynucleotides encoding one or more of: a cucurbitadienol synthase (CDS) enzyme, a C11 hydroxylase, a cytochrome P450 reductase, an epoxide hydrolase (EPH), and squalene epoxidase (SQE).

In some embodiments, the CDS enzyme comprises a sequence that is at least 90% identical to any one of SEQ ID NOs: 226, SEQ ID NO: 235, and SEQ ID NO: 232.

In some embodiments, the C11 hydroxylase comprises the sequence of any one of SEQ ID NOs: 280-281 and 305.

In some embodiments, the cytochrome P450 reductase comprises the sequence of any one of SEQ ID NOs: 282-283 and 306-307 or a sequence that has at least 90% identity to any one of SEQ ID NOs: 282-283 and 306-307.

In some embodiments, the EPH comprises the sequence of any one of SEQ ID NO: 284-292 and 309-310 or a sequence that has at least 90% identity to any one of SEQ ID NO: 284-292 and 309-310.

In some embodiments, the SQE comprises the sequence of any one of SEQ ID NOs: 293-295 and 312 or a sequence that has at least 90% identity to any one of SEQ ID NOs: 293-295 and 312.

In some embodiments, the host cell further comprises a secondary UGT. In some embodiments, the secondary UGT comprises a sequence that is at least 90% identical to any one of SEQ ID NOs: 104-106, 108-114, and 121. In some embodiments, the secondary UGT comprises the sequence of any one of SEQ ID NOs: 104-106 and 108-114.

In some embodiments, the host cell is a yeast cell, a plant cell, or a bacterial cell. In some embodiments, the host cell is a yeast cell. In some embodiments, the yeast cell is a Saccharomyces cerevisiae cell. In some embodiments, the yeast cell is a Yarrowia cell.

In some embodiments, the host cell is a bacterial cell. In some embodiments, the bacterial cell is an E. coli cell.

Further aspects of the present disclosure provide methods of producing a mogroside. In some embodiments, the methods comprise culturing any of the host cells disclosed herein with at least one mogroside precursor.

In some embodiments, the mogroside precursor is selected from mogrol, MIE1, MIA1, MIIA, MIIA1, MIIE, MIII, MIIIA1, and MIIIE.

In some embodiments, the mogroside that is produced is selected from MIA1, MIIA1, MIIE1, MIE1, MIII, MIIIA1, MIIIE, siamenoside I, and MV.

Further aspects of the present disclosure provide host cells that comprise a heterologous polynucleotide encoding a UDP-glycosyltransferase (UGT), wherein the UGT comprises a sequence that is at least 90% identical to any one of SEQ ID NOs: 104-106 and 108-114, and wherein the UGT is capable of producing one or more mogrosides from a mogroside precursor.

In some embodiments, the UGT comprises the sequence of any one of SEQ ID NOs: 104-106 and 108-114.

In some embodiments, the mogroside precursor is selected from mogrol, MIE1, MIA1, MIIA, MIIA1, MIIE, MIII, MIIIA1, and MIIIE.

In some embodiments, the mogroside that is produced is selected from MIA1, MIIA1, MIIIA1, MIIE1, MIII, siamenoside I, and MIIIE.

In some embodiments, the host cell further comprises one or more heterologous polynucleotides encoding one or more of: a cucurbitadienol synthase (CDS) enzyme, a C11 hydroxylase, a cytochrome P450 reductase, an epoxide hydrolase (EPH), and squalene epoxidase (SQE).

In some embodiments, the CDS enzyme comprises a sequence that is at least 90% identical to any one of SEQ ID NOs: 226, SEQ ID NO: 235, and SEQ ID NO: 232.

In some embodiments, the C11 hydroxylase comprises the sequence of any one of SEQ ID NOs: 280-281 and 305 or a sequence that is at least 90% identical to any one SEQ ID NOs: 280-281 and 305.

In some embodiments, the cytochrome P450 reductase comprises the sequence of any one of SEQ ID NOs: 282-283 and 306-307 or a sequence that is at least 90% identical to any one SEQ ID NOs: 282-283 and 306-307.

In some embodiments, the EPH comprises the sequence of any one of SEQ ID NOs: 284-292 and 309-310 or a sequence that is at least 90% identical to any one SEQ ID NOs: 284-292 and 309-310.

In some embodiments, the SQE comprises the sequence of any one of SEQ ID NOs: 293-295 and 312 or a sequence that is at least 90% identical to any one SEQ ID NOs: 293-295 and 312.

In some embodiments, the host cell further comprises a primary UGT. In some embodiments, the primary UGT comprises a sequence that is at least 90% identical to any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 103, 74, 79, 102, 62, 87.

In some embodiments, the primary UGT comprises the sequence of any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 103, 74, 79, 102, 62, and 87.

In some embodiments, the host cell is a yeast cell, a plant cell, or a bacterial cell.

In some embodiments, the host cell is a yeast cell. In some embodiments, the yeast cell is a Saccharomyces cerevisiae cell. In some embodiments, the yeast cell is a Yarrowia cell.

In some embodiments, the host cell has reduced or eliminated expression of EXG1.

In some embodiments, the host cell is a bacterial cell. In some embodiments, the bacterial cell is an E. coli cell.

Further aspects of the present disclosure provide methods of producing a mogroside comprising culturing any of the host cells disclosed herein at least one mogroside precursor.

In some embodiments, the mogroside precursor is selected from mogrol, MIE1, MIA1, MIIA, MIIA1, MIIE, MIII, MIIIA1, and MIIIE.

In some embodiments, the mogroside that is produced is selected from MIA1, MIIA1, MIIIA1, MIIE1, MIII, siamenoside I, and MIIIE.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIGS. 1A-1D include schematic overviews of putative mogrol biosynthesis pathways and examples of UGT activity. SQS indicates squalene synthase, EPD indicates epoxidase, P450 indicates C11 hydroxylase, EPH indicates epoxide hydrolase, and CDS indicates cucurbitadienol synthase. FIG. 1A and FIG. 1B show putative mogrol biosynthesis pathways. FIG. 1C shows non-limiting examples of primary UGT activity. FIG. 1D shows non-limiting examples of secondary UGT activity.

FIG. 2 is a graph showing liquid chromatography-mass spectrometry (LC-MS) profiles of candidate UGTs that were identified in Example 1 as having primary glycosylation activity. Average mg/L of mogrol, mogroside I-A1 (MIA1), mogroside I-E1 (MIE1), and mogroside II-E (MIIE1) for each candidate UGT is shown. A UGT that was known to have primary glycosylation activity was used as a positive control, and GFP was used as a negative control. UGT candidates were expressed by chromosomal integration in a chassis strain.

FIGS. 3A-3C depicts candidate UGTs from FIG. 2 that demonstrated considerable primary glycosylation activity. FIG. 3A depicts candidate UGTs with MIA1 (C24 glycosylation) activity. FIG. 3B depicts candidate UGTs with MIE1 (C3 glycosylation) activity. FIG. 3C depicts candidate UGTs with MIIE1 (C3 and C24 glycosylation) activity.

FIG. 4 is a graph depicting the mogroside profiles for two candidate UGTs that were identified in Example 2 as having secondary glycosylation activity. Mogroside profiles are shown as percentages of MIs, MIIs, MIIIs, MIVs, and MVs. A UGT that was known to have secondary glycosylation activity was used as a positive control, and GFP was used as a negative control. UGT candidates were expressed by chromosomal integration in a chassis strain.

FIG. 5 is a graph depicting the mogroside profiles for nine candidate UGTs that were identified in Example 3 as having secondary glycosylation activity. Mogroside profiles are shown as percentages of MIs, MIIs, MIIIs, MIVs, and MVs. A UGT that was known to have secondary glycosylation activity was used as a positive control, and GFP was used as a negative control. UGT candidates were expressed on plasmids in a chassis strain.

FIG. 6 depicts UGT taxa clustered by activity including UGTs that produce MIA1, UGTs that produced MIE1, UGTs that produced MIIE1, controls and inactive UGTs. PhyML was used to analyze the phylogeny. FigTree was used to generate the schematic.

FIG. 7 depicts UGT taxa clustered by Kingdom of Life. Unlabeled strains represent UGTs from Viridiplantae. Remaining strains include UGTs from viruses, bacteria, archaea, metazoan or are of unknown origin as illustrated. PhyML was used to analyze the phylogeny. FigTree was used to generate the schematic.

FIG. 8 is a schematic depicting m3710903 (SEQ ID NO: 65), m3953944 (SEQ ID NO: 75), m3710885 (SEQ ID NO: 62), and m3954606 (SEQ ID NO: 89) each superimposed onto the structure of a reference UGT (SEQ ID NO: 121). Rectangles are used to highlight structural differences between each UGT and the reference UGT. Circles are used to highlight sequence insertions between each UGT and the reference UGT.

FIGS. 9A-9B include graphs depicting mogroside profiles for candidate primary UGTs as described in Example 5. FIG. 9A shows the concentration in mg/L of MIA1, MIE1, MIIA1, and mogrol produced by the indicated strains. FIG. 9B shows the concentration in mg/L of MIIA1, MIIE1, MIII, MIIIA1, MIIIA2, MIIIE, siamenoside I (Sia), and MV produced by the indicated strains.

### DETAILED DESCRIPTION

Mogrosides are widely used as natural sweeteners, for example in beverages. However, de novo synthesis and mogroside extraction from natural sources often involves high production costs and low yield. This disclosure provides host cells that are engineered to efficiently produce mogrol (or 11, 24, 25-trihydroxy cucurbitadienol), mogrosides, and precursors thereof. Methods include heterologous expression of cucurbitadienol synthase (CDS) enzymes, UDP-glycosyltransferase (UGT) enzymes, C11 hydroxylase enzymes, cytochrome P450 reductase enzymes, epoxide hydrolase (EPH) enzymes, squalene epoxidase (SQE) enzymes, or combinations thereof. Examples 1-3 describe the identification of primary and secondary UGT enzymes for mogroside production. Enzymes and host cells described in this disclosure can be used for making mogrol, mogrosides, and precursors thereof.

### Synthesis of Mogrol and Mogrosides

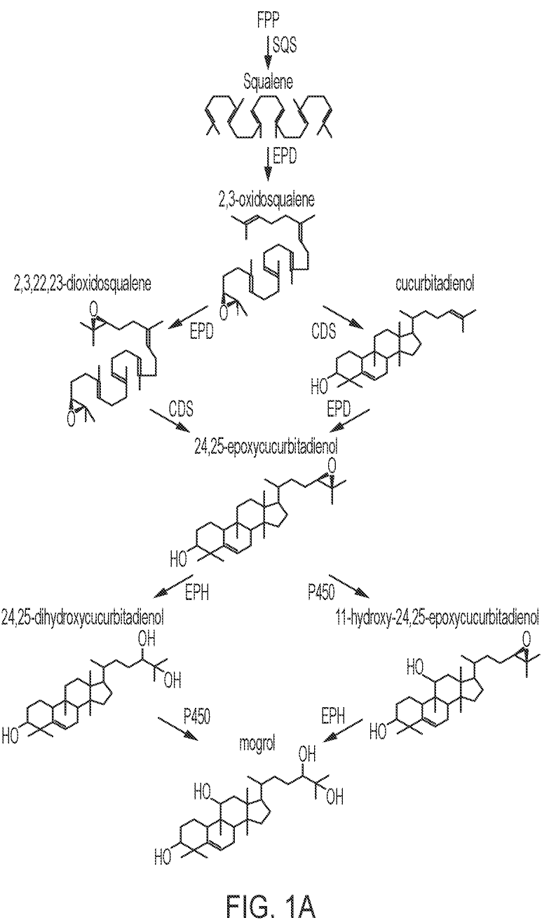

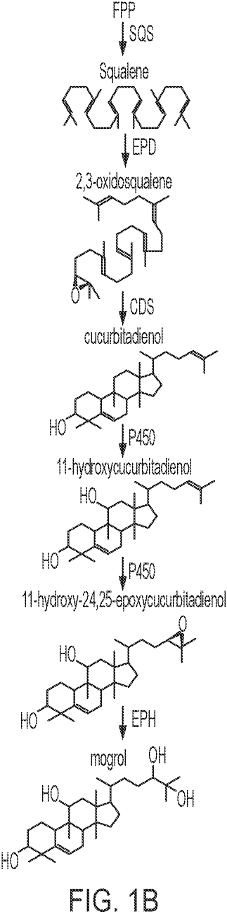

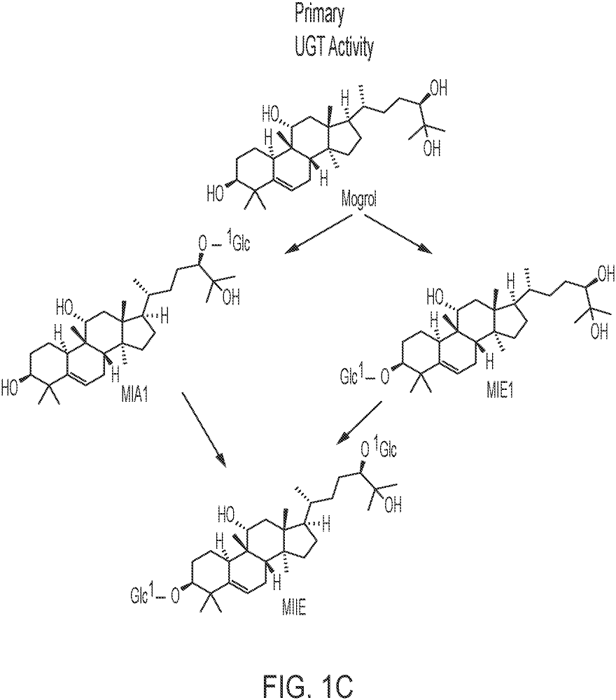

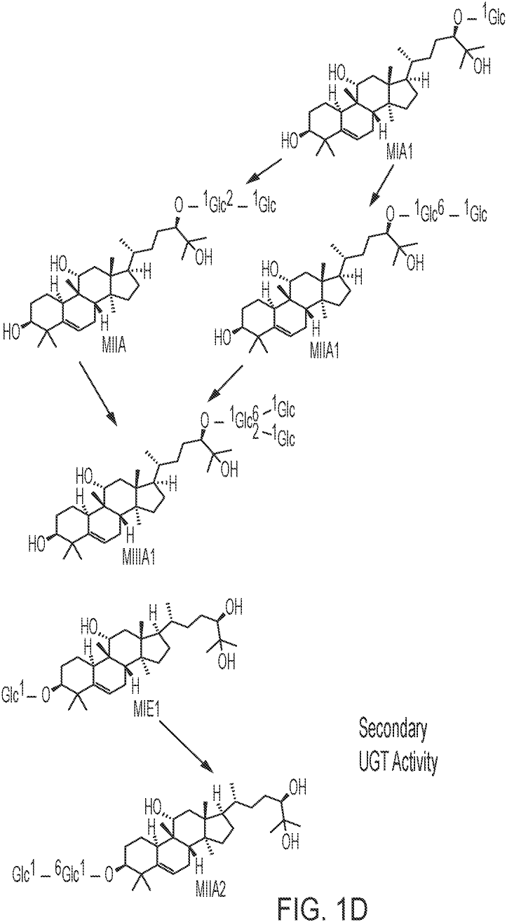

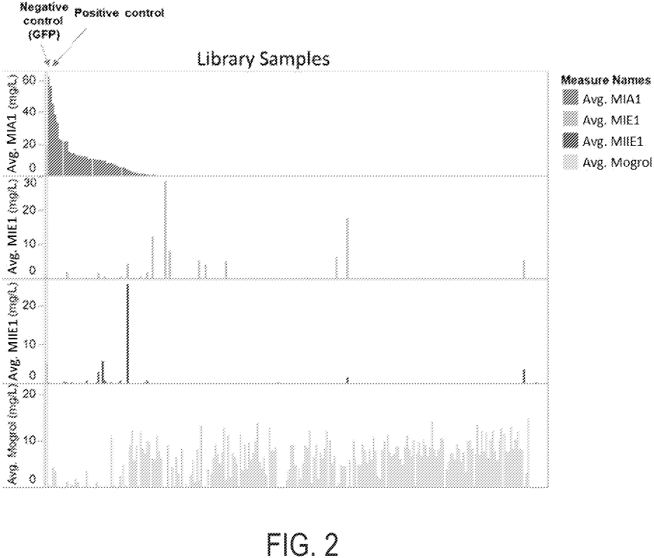

FIGS. 1A-1B show putative mogrol synthesis pathways. An early step in the pathway involves conversion of squalene to 2,3-oxidoqualene. As shown in FIGS. 1A, 2,3-oxidosqualene can be first cyclized to cucurbitadienol followed by epoxidation to form 24,25-epoxycucurbitadienol, or 2,3-oxidosqualene can be epoxidized to 2,3,22,23-dioxidosqualene and then cyclized to 24,25-epoxycucurbitadienol. Next, the 24,25-epoxycucurbitadienol can be converted to mogrol (an aglycone of mogrosides) following epoxide hydrolysis and then oxidation, or oxidation and then epoxide hydrolysis. As shown in FIGS. 1B, 2,3-oxidosqualene can be first cyclized to cucurbitadienol, which is then converted to 11-hydroxycucurbitadienol by a cytochrome P450 C11 hydroxylase. Then, a cytochrome P450 C11 hydroxylase may convert 11-hydroxycucurbitadienol to 11-hydroxy-24,25-epoxycucurbitadienol. 11-hydroxy-24,25-epoxycucurbitadienol may be converted to mogrol by epoxide hydrolase. C11 hydroxylases act in conjunction with cytochrome P450 reductases (not shown in FIGS. 1A-1B).

Mogrol can be distinguished from other cucurbitane triterpenoids by oxygenations at C3, C11, C24, and C25. Glycosylation of mogrol, for example at C3 and/or C24, leads to the formation of mogrosides.

Mogrol precursors include but are not limited to squalene, 2-3-oxidosqualene, 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxycucurbitadienol, 11-oxo-cucurbitadienol, and 24,25-dihydroxycucurbitadienol. The term “dioxidosqualene” may be used to refer to 2,3,22,23-diepoxy squalene or 2,3,22,23-dioxido squalene. The term “2,3-epoxysqualene” may be used interchangeably with the term “2-3-oxidosqualene.” As used in this application, mogroside precursors include mogrol precursors, mogrol and mogrosides.

Examples of mogrosides include, but are not limited to, mogroside I-A1 (MIA1), mogroside IE (MIE or M1E), mogroside II-A1 (MIIA1 or M2A1), mogroside II-A2 (MIIA2 or M2A2), mogroside III-A1 (MIIIA1 or M3A1), mogroside II-E (MIIE, MIIE1, or M2E), mogroside III (MIII or M3), siamenoside I (siamenoside), mogroside IV (MIV or M4), mogroside IVa (MIVA or M4A), isomogroside IV, mogroside III-E (MIIIE or M3E), mogroside V (MV or M5), and mogroside VI (MVI or M6). In some embodiments, the mogroside produced is siamenoside I, which may be referred to as Siam. In some embodiments, the mogroside produced is MIIIE. Unless otherwise noted, when used in the plural, the terms “M1s”, “MIs”, “M2s”, “MIIs”, “M3s”, “MIIIs”, “M4s”, “MIVs”, “MVs”, “M5s”, “M6s”, and “MVIs” each refer to a class of mogrosides. As a non-limiting example, M2s or MIIs may include MIIA1, MIIA, MIIA2, and/or MIIE1.

In other embodiments, a mogroside is a compound of Formula 1:

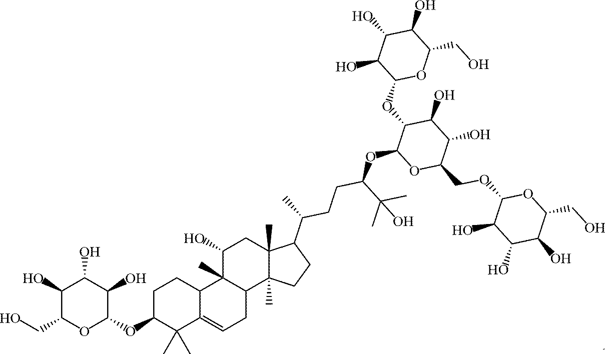

In some embodiments, the methods described in this application may be used to produce any of the compounds described in and incorporated by reference from US 2019/0071705, including compounds 1-20 as disclosed in US 2019/0071705. In some embodiments, the methods described in this application may be used to produce variants of any of the compounds described in and incorporated by reference from US 2019/0071705, including variants of compounds 1-20 as disclosed in US 2019/0071705. For example, a variant of a compound described in US 2019/0071705 can comprise a substitution of one or more alpha-glucosyl linkages in a compound described in US 2019/0071705 with one or more beta-glucosyl linkages. In some embodiments, a variant of a compound described in US 2019/0071705 comprises a substitution of one or more beta-glucosyl linkages in a compound described in US 2019/0071705 with one or more alpha-glucosyl linkages. In some embodiments, a variant of a compound described in US 2019/0071705 is a compound of Formula 1 shown above.

In some embodiments, a host cell comprising one or more enzymes described herein (e.g., a UDP-glycosyltransferase (UGT) enzyme, a cucurbitadienol synthase (CDS) enzyme, a C1 1 hydroxylase enzyme, a cytochrome P450 reductase enzyme, an epoxide hydrolase enzyme (EPH), and/or a squalene epoxidase enzyme (SQE)) is capable of producing at least at least 0.005 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, or at least 10,000 mg/L of one or more mogrosides and/or mogroside precursors. In some embodiments, the mogroside is mogroside I-A1 (MIA1), mogroside IE (MIE or M1E), mogroside II-A1 (MIIA1 or M2A1), mogroside II-A2 (MIIA2 or M2A2), mogroside III-A1 (MIIIA1 or M3A1), mogroside II-E (MIIE, MIIE1, or M2E), mogroside III (MIII or M3), siamenoside I, mogroside IV (MIV or M4), mogroside IVa (MIVA or M4A), isomogroside IV, mogroside III-E (MIIIE or M3E), mogroside V (MV or M5), or mogroside VI (MVI or M6).

### UDP-Glycosyltransferases (UGT) Enzymes

Aspects of the present disclosure provide UDP-glycosyltransferase enzymes (UGTs), which may be useful, for example, in the production of a mogroside (e.g., mogroside I-A1 (MIA1), mogroside I-E (MIE), mogroside II-A1 (MIIA1), mogroside II-A2 (MIIA2), mogroside III-A1 (MIIIA1), mogroside II-E (MIIE, MIIE1, or M2E), mogroside III (MIII), siamenoside I, mogroside III-E (MIIIE), mogroside IV, mogroside IVa, isomogroside IV, mogroside V, or mogroside VI).

As used in this disclosure, a “UGT” refers to an enzyme that is capable of catalyzing the addition of the glycosyl group from a UTP-sugar to a compound (e.g., mogroside or mogrol). A UGT may be a primary and/or a secondary UGT.

A “primary” UGT, or a UGT that has “primary glycosylation activity,” refers to a UGT that is capable of catalyzing the addition of a glycosyl group to a position on a compound that does not comprise a glycosyl group. For example, a primary UGT may be capable of adding a glycosyl group to the C3 and/or C24 position of an isoprenoid substrate (e.g., mogrol). See, e.g., FIG. 1C.

A “secondary” UGT, or a UGT that has “secondary glycosylation activity,” refers to a UGT that is capable of catalyzing the addition of a glycosyl group to a position on a compound that already comprises a glycosyl group. See, e.g., FIG. 1D. As a non-limiting example, a secondary UGT may add a glycosyl group to a mogroside I-A1 (MIA1), mogroside I-E (MIE), mogroside II-A1 (MIIA1), mogroside II-A2 (MIIA2), mogroside III-A1 (MIIIA1), mogroside II-E (MIIE, MIIE1, or M2E), mogroside III (MIII),siamenoside I, mogroside III-E (MIIIE), mogroside IV, mogroside IVa, isomogroside IV, mogroside V, and/or mogroside VI.

Example 1 describes identification of primary UGTs. In some embodiments, a primary UGT of the present disclosure comprises a sequence (e.g., nucleic acid or amino acid sequence) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a sequence in Table 8 or Table 11, or to a sequence selected from any one of SEQ ID NOs: 1-2, 4-18, 20, 22-45, 47-53, 62-63, 65-79, 81, 83-106, 108-114, 314-318, and 319-323, or to any UGT sequence disclosed in this application or known in the art.

In some embodiments, a primary UGT of the present disclosure comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a sequence selected from SEQ ID NOs: 65-73, 75, 76, 78, 81, 83-86, 88-101, 62, 74, 79, 87, 102, 103, 63, 77, 84, and 94.

In some embodiments, a primary UGT of the present disclosure comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a sequence selected from SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 103, 74, 79, 102, 62, and 87.

In some embodiments, a primary UGT is be capable of adding a glycosyl group to the C24 position of an isoprenoid substrate and comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a sequence selected from SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, and 84.

In some embodiments, a primary UGT is be capable of adding a glycosyl group to the C3 position of an isoprenoid substrate and comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a sequence selected from SEQ ID NOs: 103, 74, 79, 102, 62, and 87.

In some embodiments, a primary UGT is be capable of adding a glycosyl group to the C3 position of an isoprenoid substrate and comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a sequence selected from SEQ ID NOs: 103, 74, 79, 102, 62, 87, and 63.

In some embodiments, a primary UGT comprises a sequence that is at least 90% identical to any one of SEQ ID NOs: 65-73, 75, 76, 78, 81, 83-86, and 88-101, and is capable of catalyzing conversion of mogrol to MIA1. In some embodiments, the primary UGT comprises the sequence of any one of SEQ ID NOs: 65-73, 75, 76, 78, 81, 83-86, and 88-101.

In some embodiments, a primary UGT comprises a sequence that is at least 90% identical to any one of SEQ ID NOs: 62, 74, 79, 87, 102, and 103, and is capable of catalyzing conversion of mogrol to MIE1. In some embodiments, the primary UGT comprises the sequence of any one of SEQ ID NOs: 62, 74, 79, 87, 102, and 103.

In some embodiments, a primary UGT comprises a sequence that is at least 90% identical to any one of SEQ ID NOs: 63, 77, 84, and 94, and is capable of catalyzing conversion of mogrol to MIIE1. In some embodiments, the primary UGT comprises the sequence of any one of SEQ ID NOs: 63, 77, 84, and 94.

In some embodiments, a primary UGT comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 65, 66, or 68. In some embodiments, a sequence that is at least 80% identical to SEQ ID NO: 65 is SEQ ID NO: 88 or SEQ ID NO: 66. In some embodiments, a sequence that is at least 90%, at least 91%, at least 92%, at least 93%, at least 94%, at least 95%, at least 96%, at least 97%, at least 98%, or at least 99% identical to SEQ ID NO: 66 is SEQ ID NO: 88.

In some embodiments, a primary UGT comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 96 or 97. In some embodiments, a sequence that is at least 80%, at least 81%, at least 82%, at least 83%, at least 84%, at least 85%, at least 86%, or at least 87% identical to SEQ ID NO: 96 is SEQ ID NO: 97.

In some embodiments, a primary UGT comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 92 or 93. In some embodiments, a sequence that is at least 80% identical to SEQ ID NO: 92 is SEQ ID NO: 93.

In some embodiments, a primary UGT comprises a sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 68 or 71. In some embodiments, a sequence that is at least 70% identical to SEQ ID NO: 68 is SEQ ID NO: 71. In some embodiments, a sequence that is at least 75% identical to SEQ ID NO: 68 is SEQ ID NO: 71.

In some embodiments, a primary UGT comprises a sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 70 or 76. In some embodiments, a sequence that is at least 70% identical to SEQ ID NO: 70 is SEQ ID NO: 76. In some embodiments, a sequence that is at least 75% identical to SEQ ID NO: 70 is SEQ ID NO: 76.

In some embodiments, a primary UGT comprises a sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 85, 95, 86, 81, 96, or 97. In some embodiments, a sequence that is at least 70% identical to SEQ ID NO: 96 is SEQ ID NO: 85, 95, 86, 81, or 97. In some embodiments, a sequence that is at least 75% identical to SEQ ID NO: 81 is 85, 95, 86, 96, or 97.

In some embodiments, a primary UGT comprises a sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 90 or 91. In some embodiments, a sequence that is at least 70% identical to SEQ ID NO: 90 is SEQ ID NO: 91.

Examples 2 and 3 describe identification of secondary UGTs. In some embodiments, a secondary UGT of the present disclosure comprises a sequence (e.g., nucleic acid or amino acid sequence) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a sequence in Table 8 or Table 11, or to a sequence selected from SEQ ID NOs: 1-2, 4-18, 20, 22-45, 47-53, 62-63, 65-79, 81, 83-106, 108-114, and 121, or to any UGT sequence disclosed in this application or known in the art.

In some embodiments, a secondary UGT comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a sequence selected from SEQ ID NOs: 104-106 and 108-114.

The UGTs of the present disclosure may be capable of glycosylating mogrol or a mogroside at any of the oxygenated sites (e.g., at C3, C11, C24, and C25). In some embodiments, the UGT is capable of branching glycosylation (e.g., branching glycosylation of a mogroside at C3 or C24).

Non-limiting examples of suitable substrates for the UGTs of the present disclosure include mogrol and mogrosides (e.g., mogroside IA1 (MIA1), mogroside IE (MIE), mogroside II-A1 (MIIA1), mogroside III-A1 (MIIIA1), mogroside II-E (MIIE or MIIE1), mogroside III (MIII),or mogroside III-E (MIIIE), siamenoside I).

In some embodiments, the UGTs of the present disclosure are capable of producing mogroside IA1 (MIA1), mogroside IE (MIE), mogroside II-A1 (MIIA1), mogroside II-A2 (MIIA2), mogroside III-A1 (MIIIA1), mogroside II-E (MIIE or MIIE1), mogroside III (MIII),siamenoside I, mogroside III-E (MIIIE), mogroside IV, mogroside IVa, isomogroside IV, and/or mogroside V.

In some embodiments, the UGT is capable of catalyzing the conversion of mogrol to MIA1; mogrol to MIE1; MIA1 to MIIA1; MIE1 to MIIE1; MIIA1 to MIIIA1; MIA1 to MIIE1; MIIA1 to MIII; MIIIA1 to siamenoside I; MIIE1 to MIII; MIII to siamenoside I; MIIE1 to MIIIE; and/or MIIIE to siamenoside I.

It should be appreciated that activity, such as specific activity, of a UGT can be measured by any means known to one of ordinary skill in the art. In some embodiments, the activity, such as specific activity, of a UGT may be determined by measuring the amount of glycosylated mogroside produced per unit enzyme per unit time. For example, the activity, such as specific activity, may be measured in mmol glycosylated mogroside target produced per gram of enzyme per hour. In some embodiments, a UGT of the present disclosure may have an activity, such as specific activity, of at least 0.1 mmol (e.g., at least 1 mmol, at least 1.5 mmol, at least 2 mmol, at least 2.5 mmol, at least 3, at least 3.5 mmol, at least 4 mmol, at least 4.5 mmol, at least 5 mmol, at least 10 mmol, including all values in between) glycosylated mogroside target produced per gram of enzyme per hour.

In some embodiments, the activity, such as specific activity, of a UGT of the present disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control UGT. In some embodiments, the control UGT is a primary UGT. In some embodiments, the control UGT is a secondary UGT. In some embodiments, the control UGT is UGT94-289-1 (a wildtype UGT sequence from the monk fruit Siraitia grosvenorii provided by SEQ ID NO: 121). In some embodiments, for a UGT that has an amino acid substitution, a control UGT is the same UGT but without the amino acid substitution.

It should be appreciated that one of ordinary skill in the art would be able to characterize a protein as a UGT enzyme based on structural and/or functional information associated with the protein. For example, a protein can be characterized as a UGT enzyme based on its function, such as the ability to produce one or more mogrosides in the presence of a mogroside precursor, such as mogrol.

A UGT enzyme can be further characterized as a primary UGT based on its function of catalyzing the addition of a glycosyl group to a position on a compound that does not comprise a glycosyl group. A UGT enzyme can be characterized as a secondary UGT based on its function of catalyzing the addition of a glycosyl group to a position on a compound that already comprises a glycosyl group. In some embodiments, a UGT enzyme can be characterized as a both primary and a secondary UGT enzyme.

In other embodiments, a protein can be characterized as a UGT enzyme based on the percent identity between the protein and a known UGT enzyme. For example, the protein may be at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical, including all values in between, to any of the UGT sequences described in this application or the sequence of any other UGT enzyme.

In other embodiments, a protein can be characterized as a UGT enzyme based on the presence of one or more domains in the protein that are associated with UGT enzymes. For example, in certain embodiments, a protein is characterized as a UGT enzyme based on the presence of a sugar binding domain and/or a catalytic domain, characteristic of UGT enzymes known in the art. In certain embodiments, the catalytic domain binds the substrate to be glycosylated.

In other embodiments, a protein can be characterized as a UGT enzyme based on a comparison of the three-dimensional structure of the protein compared to the three-dimensional structure of a known UGT enzyme. For example, a protein could be characterized as a UGT based on the number or position of alpha helical domains, beta-sheet domains, etc. It should be appreciated that a UGT enzyme can be a synthetic protein.

Structurally, UGTs often comprise a UDPGT (Prosite: PS00375) domain and a catalytic dyad. As a non-limiting example, one of ordinary skill in the art may identify a catalytic dyad in a UGT by aligning the UGT sequence to UGT94-289-1 and identifying the two residues in the UGT that correspond to histidine 21 (H21) and aspartate 122 (D122) of UGT94-289-1.

The amino acid sequence for UGT94-289-1 is:

MDAQRGHTTTILMFPWLGYGHLSAFLELAKSLSRRNFHIYFCSTSVNLDAIKP
KLPSSSSSDSIQLVELCLPSSPDQLPPHLHTTNALPPHLMPTLHQAFSMAAQHFAAILH
TLAPHLLIYDSFQPWAPQLASSLNIPAINFNTTGASVLTRMLHATHYPSSKFPISEFVL
HDYWKAMYSAAGGAVTKKDHKIGETLANCLHASCSVILINSFRELEEKYMDYLSVL
LNKKVVPVGPLVYEPNQDGEDEGYSSIKNWLDKKEPSSTVFVSFGSEYFPSKEEMEEI
AHGLEASEVHFIWVVRFPQGDNTSAIEDALPKGFLERVGERGMVVKGWAPQAKILK
HWSTGGFVSHCGWNSVMESMMFGVPIIGVPMHLDQPFNAGLAEEAGVGVEAKRDP
DGKIQRDEVAKLIKEVVVEKTREDVRKKAREMSEILRSKGEEKMDEMVAAISLFLKI
(SEQ ID NO: 121).

A non-limiting example of a nucleic acid sequence encoding UGT94-289-1 is:

atggacgcgcaacgcggacatacgactaccatcctgatgtttccgtggttggggtacggccaccttagtgcattcctcgaatt
agccaagagcttgtcgcgtaggaactttcatatttatttctgttccacatctgtcaatttagatgctataaaacccaaactaccatcatcttca
agttccgattctattcagcttgtagagttatgcttgccttcctcgccagaccaactacccccacacctgcatacaactaatgctctacctcc
acatctaatgcctaccctgcaccaggccttttcaatggcagctcaacattttgcagctatattacatactttagcaccgcacttgttaatctat
gattcgttccagccttgggcgccacaattggccagctctcttaacattcctgctattaattttaataccacgggtgccagtgtgctaacaag
aatgttacacgcgactcattacccatcttcaaagttcccaatctccgaatttgttttacatgattattggaaagcaatgtattcagcagctggt
ggtgctgttacaaaaaaggaccataaaataggagaaaccttggcaaactgtttacacgcttcttgctcggtaattctgatcaattcattcag
agagttggaagaaaaatacatggattacttgtctgtcttactaaacaagaaagttgtgcccgtgggtccgcttgtttatgagccaaaccaa
gatggcgaagacgaaggttatagttcgataaagaattggctcgataaaaaggagccctcctcaactgtctttgtttccttcgggtccgaat
attttccgtccaaagaagaaatggaagaaattgcccatggcttggaggctagcgaggtacactttatttgggtcgttagattcccacaag
gagacaatacttctgcaattgaagatgcccttcctaagggttttcttgagcgagtgggcgaacgtggaatggtggttaagggttgggctc
ctcaggccaaaattttgaaacattggagcacaggcggtttcgtaagtcattgtggatggaatagtgttatggagagcatgatgtttggtgt
acccataataggtgttccgatgcatttagatcaaccatttaatgcagggctcgcggaagaagcaggagtaggggtagaggctaaaagg
gaccctgatggtaagatacagagagatgaagtcgctaaactgatcaaagaagtggttgtcgaaaaaacgcgcgaagatgtcagaaag
aaggctagggaaatgtctgaaattttacgttcgaaaggtgaggaaaagatggacgagatggttgcagccattagtctcttcttgaagata
taa (SEQ ID NO: 60).

One of ordinary skill in the art would readily recognize how to determine for any UGT enzyme what amino acid residue corresponds to a specific amino acid residue in a reference UGT such as UGT94-289-1 (SEQ ID NO: 121) by, for example, aligning sequences and/or by comparing secondary or tertiary structures.

In certain embodiments, a UGT of the present disclosure comprises one or more structural motifs corresponding to a structural motif in wild-type UGT94-289-1 (e.g., corresponding to a structural motif that is shown in Table 1). In some embodiments, a UGT comprises structural motifs corresponding to all structural motifs in Table 1. In some embodiments, a UGT comprises a structural motif that corresponds to some but not all structural motifs shown in Table 1. In some embodiments, some structural motifs may diverge by having different lengths or different helicity. For example, a UGT of the present disclosure may comprise extended versions of loops 11, 16, 20, or a combination thereof. A UGT of the present disclosure may comprise loops that have greater helicity than their counterpart in UGT94-289-1 (e.g., loops 11, 16, 20, or a combination thereof in UGT94-289-1).

TABLE 1

Non-limiting Examples of Structural Motifs in Reference Sequence UGT94-289-1 (SEQ ID NO: 121)

| Structural Motif | Borders | Sequence | SEQ ID NO |
|---|---|---|---|
| Loop 1 | Metl-Thr9 | MDAQRGHTT | 122 |
| Beta Sheet 1 | Thr10-Phe14 | TILMF | 123 |
| Loop 2 | Pro15-Gly18 | PWLG | 124 |
| Alpha Helix 1 | Tyrl9-Arg34 | YGHLSAFLELAKSLSR | 125 |
| Loop3 | Arg35-Phe37 | RNF | 126 |
| Beta Sheet 2 | His38-Phe41 | HIYF | 127 |
| Loop 4 | Cys42-Thr44 | CST | 128 |
| Alpha Helix 2 | Ser45-A1a50 | SVNLDA | 129 |
| Loop 5 | Ile51-Ser61 | IKPKLPSSSSS | 130 |
| Beta Sheet 3 | Asp62-Gln65 | DSIQ | 131 |
| Loop 6 | Leu66-Leu88 | LVELCLPSSPDQLPPHLHTTNAL | 132 |
| Alpha Helix 3 | Pro89-A1a109 | PPHLMPTLHQAFSMAAQHFAA | 133 |
| Loop 7 | Ile110-His117 | ILHTLAPH | 134 |
| Beta Sheet 4 | Leu118-Asp122 | LLIYD | 135 |
| Loop 8 | Ser123-Pro126 | SFQP | 136 |
| Alpha Helix 4 | Trp127-Leu134 | WAPQLASSL | 137 |
| Loop 9 | Asn135-Pro137 | NIP | 138 |
| Beta Sheet 5 | Ala138-Asn143 | AINFN | 139 |
| Loop 10 | Thr144-Gly146 | TTG | 140 |
| Alpha Helix 5 | Ala147-His158 | ASVLTRMLHATH | 141 |
| Loop 11 | Tyr159-Tyr179 | YPSSKFPISEFVLHDYWKAMY | 142 |
| Alpha Helix 6 | Ser180-Gly183 | SAAG | 143 |
| Loop 12 | Glyl84-Lysl89 | GAVTKK | 144 |
| Alpha Helix 7 | Asp190-Ser204 | DHKIGETLANCLHAS | 145 |
| Loop 13 | Cys205-Ser206 | CS | 146 |
| Beta Sheet 6 | Val207-Ile210 | VILI | 147 |
| Loop 14 | Asn211-Glu217 | NSFRELE | 148 |
| Alpha Helix 8 | Glu218-Leu227 | EKYMDYLSVL | 149 |
| Loop 15 | Leu228-Asn229 | LN | 150 |
| Beta Sheet 7 | Lys230-Val232 | KKV | 151 |
| Loop 16 | Val233-Ser252 | VPVGPLVYEPNQDGEDEGYS | 152 |
| Alpha Helix 9 | Ser253-Lys261 | SIKNWLDKK | 153 |
| Loop 17 | Glu262-Ser265 | EPSS | 154 |
| Beta Sheet 8 | Thr266-Ser270 | TVFVS | 155 |
| Loop 18 | Phe271-Ser278 | FGSEYFPS | 156 |
| Alpha Helix 10 | Lys279-Ser292 | KEEMEEIAHGLEAS | 157 |
| Loop 19 | Glu293-His295 | EVH | 158 |
| Beta Sheet 9 | Phe296-Val300 | FIWVV | 159 |
| Alpha Helix 11 | Arg301-Asn307 | RFPQGDN | 160 |
| Loop 20 | Thr308-Gly318 | TSAIEDALPKG | 161 |
| Alpha Helix 12 | Phe319-Val323 | FLERV | 162 |
| Loop 21 | Gly324-Gly327 | GERG | 163 |
| Beta Sheet 10 | Met328-Lys331 | MVVK | 164 |
| Loop 22 | Gly332-Pro335 | GWAP | 165 |
| Alpha Helix 13 | Gln336-Lys341 | QAKILK | 166 |
| Loop 23 | His342-Gly346 | HWSTG | 167 |
| Beta Sheet 11 | Gly347-Ser350 | GFVS | 168 |
| Loop 24 | His351-Gly353 | HCG | 169 |
| Alpha Helix 14 | Trp354-Phe363 | WNSVMESMMF | 170 |
| Loop 25 | Gly364-Pro366 | GVP | 171 |
| Beta Sheet 12 | Ile367-Val370 | IIGV | 172 |
| Loop 26 | Pro371-Leu374 | PMHL | 173 |
| Alpha Helix 15 | Asp375-Ala386 | DQPFNAGLAEEA | 174 |
| Loop 27 | Gly387-Val388 | GV | 175 |
| Beta Sheet 13 | Gly389-Glu391 | GVE | 176 |
| Loop 28 | Ala392-Gln401 | AKRDPDGKIQ | 177 |
| Alpha Helix 16 | Arg402-Val414 | RDEVAKLIKEVVV | 178 |
| Loop 29 | Glu415 | E | 179 |
| Alpha Helix 17 | Lys416-Gly436 | KTREDVRKKAREMSEILRSKG | 180 |
| Loop 30 | Glu437-Met440 | EEKM | 181 |
| Alpha Helix 18 | Asp441-Leu451 | DEMVAAISLFL | 182 |
| Loop 31 | Lys452-Ile453 | KI | 183 |

In some embodiments, a UGT is a circularly permutated version of a reference UGT. In some embodiments, a UGT comprises a sequence that includes at least two motifs from Table 1 in a different order than a reference UGT. For example, if a reference UGT comprises a first motif that is located C-terminal to a second motif, the first motif may be located N-terminal to the second motif in a circularly permutated UGT.

A UGT may comprise one or more motifs selected from Loop 1, Beta Sheet 1, Loop 2, Alpha Helix 1, Loop 3, Beta Sheet 2, Loop 4, Alpha Helix 2, Loop 5, Beta Sheet 3, Loop 6, Alpha Helix 3, Loop 7, Beta Sheet 4, Loop 8, Alpha Helix 4, Loop 9, Beta Sheet 5, Loop 10, Alpha Helix 5, Loop 11, Alpha Helix 6, Loop 12, Alpha Helix 7, Loop 13, Beta Sheet 6, Loop 14, Alpha Helix 8, and Loop 15 from Table 1 located C-terminal to one or more motifs corresponding to one or more motifs selected from Beta Sheet 7, Loop 16, Alpha Helix 9, Loop 17, Beta Sheet 8, Loop 18, Alpha Helix 10, Loop 19, Beta Sheet 9, Alpha Helix 11, Loop 20, Alpha Helix 12, Loop 21, Beta Sheet 10, Loop 22, Alpha Helix 13, Loop 23, Beta Sheet 11, Loop 24, Alpha Helix 14, Loop 25, Beta Sheet 12, Loop 26, Alpha Helix 15, Loop 27, Beta Sheet 13, Loop 28, Alpha Helix 16, Loop 29, Alpha Helix 17, Loop 30, Alpha Helix 18, and Loop 31 in Table 1.

In some embodiments, the N-terminal portion of a UGT comprises a catalytic site, including a catalytic dyad, and/or a substrate-binding site. In some embodiments, the C-terminal portion of a UGT comprises a cofactor-binding site. Aspects of the disclosure include UGTs that have been circularly permutated. In some embodiments, in a circularly permutated version of a UGT, the N-terminal portion and the C-terminal portions may be reversed in whole or in part. For example, the C-terminal portion of a circularly permutated UGT may comprise a catalytic site, including a catalytic dyad, and/or a substrate-binding site, while the N-terminal portion may comprise a cofactor-binding site. In some embodiments, a circularly permutated version of a UGT comprises a heterologous polynucleotide encoding a UGT, wherein the UGT comprises: a catalytic dyad and a cofactor binding site, wherein the catalytic dyad is located C-terminal to the cofactor-binding site.

A circularly permutated UGT encompassed by the disclosure may exhibit different properties from the same UGT that has not undergone circular permutation. In some embodiments, a host cell expressing such a circularly permutated version of a UGT produces in the presence of at least one mogroside precursor at least 10%, 20%, 30%, 40%, 50%, 60%, 70%, 80%, 90%, or 100% more of one or more mogrosides relative to a host cell that comprises a heterologous polynucleotide encoding a reference UGT that is not circularly permutated, such as wild-type UGT94-289-1 (SEQ ID NO: 121). In some embodiments, a host cell expressing such a circularly permutated version of a UGT produces in the presence of at least one mogroside precursor at least 10%, 20%, 30%, 40%, 50%, 60%, 70%, 80%, 90%, or 100% less of one or more mogrosides relative to a host cell that comprises a heterologous polynucleotide encoding a reference UGT that is not circularly permutated, such as wild-type UGT94-289-1 (SEQ ID NO: 121).

In some embodiments, a UGT of the present disclosure may comprise an amino acid substitution at an amino acid residue corresponding to an amino acid residue in wild-type UGT94-289-1 (SEQ ID NO: 121). For example, an amino acid residue that contains a substitution can be an amino acid that corresponds to an amino acid residue in wild-type UGT94-289-1 (SEQ ID NO: 121) selected from, e.g., S123; F124; N143; T144; T145; V149; Y179; G18; S180; A181; G184; A185; V186; T187; K189; Y19; H191; K192; G194; E195; A198; F276; N355; H373; L374; N47; H83; T84; T85; N86; P89; and/or L92. Non-limiting examples of such amino acid substitutions include: S123 may be mutated to alanine, cysteine, glycine or valine, or to any conservative substitution of alanine, cysteine, glycine or valine; F124 may be mutated to tyrosine or to any conservative substitution of tyrosine; N143 may be mutated to alanine, cysteine, glutamate, isoleucine, leucine, methionine, glutamine, serine, threonine or valine, or to any conservative substitution of alanine, cysteine, glutamate, isoleucine, leucine, methionine, glutamine, serine, threonine or valine; T144 may be mutated to alanine, cysteine, asparagine or proline, or to any conservative substitution of alanine, cysteine, asparagine or proline; T145 may be mutated to alanine, cysteine, glycine, methionine, asparagine, glutamine, or serine, or any conservative substitution of alanine, cysteine, glycine, methionine, asparagine, glutamine, or serine; V149 may be mutated to cysteine, leucine or methionine, or to any conservative substitution of cysteine, leucine or methionine; Y179 may be mutated to glutamate, phenylalanine, histidine, isoleucine, lysine, leucine, valine, or tryptophan, or to any conservative substitution glutamate, phenylalanine, histidine, isoleucine, lysine, leucine, valine, or tryptophan; G18 may be mutated to serine or to any conservative substitution of serine; S180 may be mutated to alanine or valine, or to any conservative substitution of alanine or valine; A181 may be mutated to lysine or threonine, or to any conservative substitution of lysine or threonine; G184 may be mutated to alanine, cysteine, aspartate, glutamate, phenylalanine, histidine, isoleucine, lysine, methionine, asparagine, proline, glutamine, arginine, serine, threonine, or tyrosine, or to any conservative substitution of alanine, cysteine, aspartate, glutamate, phenylalanine, histidine, isoleucine, lysine, methionine, asparagine, proline, glutamine, arginine, serine, threonine, or tyrosine; A185 may be mutated to cysteine, aspartate, glutamate, glycine, lysine, leucine, methionine, asparagine, proline, glutamine, threonine, tryptophan or tyrosine, or to any conservative substitution of cysteine, aspartate, glutamate, glycine, lysine, leucine, methionine, asparagine, proline, glutamine, threonine, tryptophan or tyrosine; V186 may be mutated to alanine, cysteine, aspartate, glutamate, glycine, isoleucine, lysine, leucine, methionine, asparagine, proline, glutamine, arginine, threonine, tryptophan, or tyrosine, or to any conservative substitution of alanine, cysteine, aspartate, glutamate, glycine, isoleucine, lysine, leucine, methionine, asparagine, proline, glutamine, arginine, threonine, tryptophan, or tyrosine; T187 may be mutated to alanine, cysteine, aspartate, glutamate, glycine, histidine, isoleucine, lysine, leucine, asparagine, proline, arginine, serine, valine, tryptophan, or tyrosine, or to any conservative substitution of alanine, cysteine, aspartate, glutamate, glycine, histidine, isoleucine, lysine, leucine, asparagine, proline, arginine, serine, valine, tryptophan, or tyrosine; K189 may be mutated to alanine, cysteine, aspartate, glutamate, phenylalanine, glycine, histidine, isoleucine, leucine, methionine, proline, glutamine, arginine, serine, threonine, valine, tryptophan, or tyrosine, or to any conservative substitution thereof of alanine, cysteine, aspartate, glutamate, phenylalanine, glycine, histidine, isoleucine, leucine, methionine, proline, glutamine, arginine, serine, threonine, valine, tryptophan, or tyrosine; Y19 may be mutated to phenylalanine, histidine, leucine, or valine, or to any conservative substitution of phenylalanine, histidine, leucine, or valine; H191 may be mutated to alanine, cysteine, aspartate, glutamate, glycine, lysine, methionine, proline, glutamine, serine, threonine, valine, tryptophan, or tyrosine, or to any conservative substitution of mutated to alanine, cysteine, aspartate, glutamate, glycine, lysine, methionine, proline, glutamine, serine, threonine, valine, tryptophan, or tyrosine; K192 may be mutated to cysteine or phenylalanine, or to any conservative substitution of cysteine or phenylalanine; G194 may be mutated to aspartate, leucine, methionine, asparagine, proline, serine, or tryptophan, or to any conservative substitution of aspartate, leucine, methionine, asparagine, proline, serine, or tryptophan; E195 may be mutated to alanine, isoleucine, lysine, leucine, asparagine, glutamine, serine, threonine, or tyrosine, or to any conservative substitution of alanine, isoleucine, lysine, leucine, asparagine, glutamine, serine, threonine, or tyrosine; A198 may be mutated to cysteine, aspartate, glutamate, phenylalanine, histidine, isoleucine, lysine, leucine, methionine, asparagine, proline, glutamine, arginine, serine, threonine, valine, or tyrosine, or to any conservative substitution of cysteine, aspartate, glutamate, phenylalanine, histidine, isoleucine, lysine, leucine, methionine, asparagine, proline, glutamine, arginine, serine, threonine, valine, or tyrosine; F276 may be mutated to cysteine or glutamine, or to any conservative substitution of cysteine or glutamine; N355 may be mutated to glutamine or serine, or any conservative substitution thereof; H373 may be mutated to lysine, leucine, methionine, arginine, valine, or tyrosine, or to any conservative substitution of lysine, leucine, methionine, arginine, valine, or tyrosine; L374 may be mutated to alanine, cysteine, phenylalanine, histidine, methionine, asparagine, glutamine, serine, threonine, valine, tryptophan, or tyrosine, or to any conservative substitution of alanine, cysteine, phenylalanine, histidine, methionine, asparagine, glutamine, serine, threonine, valine, tryptophan, or tyrosine; N47 may be mutated to glycine or to any conservative substitution of glycine; H83 may be mutated to glutamine or tryptophan, or to any conservative substitution of glutamine or tryptophan; T84 may be mutated to tyrosine or to any conservative substitution of tyrosine; T85 may be mutated to glycine, lysine, proline, serine, or tyrosine, or to any conservative substitution of glycine, lysine, proline, serine, or tyrosine; N86 may be mutated to alanine, cysteine, glutamate, isoleucine, lysine, leucine, serine, tryptophan, or tyrosine, or to any conservative substitution of alanine, cysteine, glutamate, isoleucine, lysine, leucine, serine, tryptophan, or tyrosine; P89 may be mutated to methionine or serine or to any conservative substitution of methionine or serine; and/or L92 may be mutated to histidine or lysine or to any conservative substitution of histidine or lysine.

A UGT of the present disclosure can comprise a conservative amino acid substitution and/or a non-conservative amino acid substitution. In some embodiments, a UGT of the present disclosure comprises 1, 2, 3, 4, 5, 6, 7, 8, 9, 10 or more than 10 conservative amino acid substitution(s). In some embodiments, a UGT of the present disclosure comprises 1, 2, 3, 4, 5, 6, 7, 8, 9, 10 or more than 10 non-conservative amino acid substitutions. In some embodiments, a conservative or non-conservative amino acid substitution is not located in a conserved region of a UGT protein. In some embodiments, a conservative or non-conservative amino acid substitution is not located in a region corresponding to: residues 83 to 92; residues 179 to 198; residue N143; residue L374; residue H21; or residue D122 of wild-type UGT94-289-1. One of ordinary skill in the art would readily be able to test a UGT that comprises a conservative and/or non-conservative substitution to determine whether the conservative and/or non-conservative substitution impacts the activity or function of the UGT.

In some embodiments, a UGT enzyme contains an amino acid substitution located within 10 angstrom, 9 angstrom 8 angstrom, 7 angstrom, 6 angstrom, 5 angstrom, 4 angstrom, 3 angstrom, 2 angstrom, or within 1 angstrom (including all values in between) of a catalytic dyad. The catalytic dyad may correspond to residues 21 and 122 of wildtype UGT94-289-1 (e.g., histidine 21 and aspartate acid 122). It should be appreciated that one of ordinary skill in the art would readily recognize how to determine to corresponding location of the catalytic dyad in any UGT enzyme, for example, by aligning the sequence and/or by comparing the secondary structure with UGT94-289-1 (SEQ ID NO: 121).

In some embodiments, a UGT enzyme contains an amino acid substitution at an amino acid residue located in one or more structural motifs of the UGT. Non-limiting examples of secondary structures in UGTs, such as UGT94-289-1 (SEQ ID NO: 121), include: the loop between beta sheet 4 and alpha helix 5; beta sheet 5; the loop between beta sheet 5 and alpha helix 6; alpha helix 6; the loop between alpha helix 6 & 7; the loop between beta sheet 1 & alpha helix 1; alpha helix 7; the loop between alpha helix 7 & 8; alpha helix 1; alpha helix 8; the loop between beta sheet 8 & alpha helix 13; alpha helix 17; the loop between beta sheet 12 & alpha helix 18; alpha helix 2; loop between beta sheet 3 & alpha helix 3; alpha helix 3; and the loop between alpha helix 3 & 4; loop 8; beta sheet 5; loop 10; alpha helix 5; loop 11; loop 2; alpha helix 6; loop 12; alpha helix 1; alpha helix 7; loop 18; alpha helix 14; loop 26; alpha helix 2; loop 6; and alpha helix 3.

In some embodiments: the amino acid residue located in loop 8 is a residue corresponding to S123 or F124 in UGT94-289-1 (SEQ ID NO: 121); the amino acid residue located in beta sheet 5 is a residue corresponding to N143 in UGT94-289-1 (SEQ ID NO: 121); the amino acid residue located in loop 10 is a residue corresponding to T144 or T145 in UGT94-289-1 (SEQ ID NO: 121); the amino acid residue located in alpha helix 5 is a residue corresponding to V149 in UGT94-289-1 (SEQ ID NO: 121); the amino acid residue located in loop 11 is a residue corresponding to Y179 in UGT94-289-1 (SEQ ID NO: 121); the amino acid residue located in loop 2 is a residue corresponding to G18 in UGT94-289-1 (SEQ ID NO: 121); the amino acid residue located in alpha helix 6 is a residue corresponding to S180 or A181 in UGT94-289-1 (SEQ ID NO: 121); the amino acid residue located in loop 12 is a residue corresponding to G184, A185, V186, T187, or K189 in UGT94-289-1 (SEQ ID NO: 121); the amino acid residue located in alpha helix 1 is a residue corresponding to Y19 in UGT94-289-1 (SEQ ID NO: 121); the amino acid residue located in alpha helix 7 is a residue corresponding to H191, K192, G194, E195, or A198 in UGT94-289-1 (SEQ ID NO: 121); the amino acid residue located in loop 18 is a residue corresponding to F276 in UGT94-289-1 (SEQ ID NO: 121); the amino acid residue located in alpha helix 14 is a residue corresponding to N355 in UGT94-289-1 (SEQ ID NO: 121); the amino acid residue located in loop 26 is a residue corresponding to H373 or L374 in UGT94-289-1 (SEQ ID NO: 121); the amino acid residue located in alpha helix 2 is a residue corresponding to N47 in UGT94-289-1 (SEQ ID NO: 121); the amino acid residue located in loop 6 is a residue corresponding to H83, T84, T85, or N86 in UGT94-289-1 (SEQ ID NO: 121); and/or the amino acid residue located in alpha helix 3 is a residue corresponding to P89 or L92 in UGT94-289-1 (SEQ ID NO: 121).

In some embodiments, a UGT comprises an amino acid substitution (e.g., at least 1, 2, 3, 4, 5, 6, 7, or 8 substitutions) in a region corresponding to residues 83 to 92, 179 to 189, 1 to 82, 93 to 142, 144 to 178, 199 to 373, or 375 to 453 of UGT94-289-1 (SEQ ID NO: 121). In some embodiments, the UGT comprises an amino acid substitution at an amino acid residue corresponding to the amino acid residue in wild-type UGT94-289-1 (SEQ ID NO: 121) selected from: N143 and L374. In some embodiments, the residue corresponding to N143 is mutated to a negatively charged R group, a polar uncharged R group, or a nonpolar aliphatic R group. In some embodiments, the residue corresponding to L374 is mutated to a nonpolar aliphatic R group, a positively charged R group, a polar uncharged R group, or a nonpolar aromatic R group. In some embodiments, a UGT comprises a region that is at least 90% identical to residues 83 to 92 of UGT94-289-1 or at least 95% identical to residues 179 to 198 of UGT94-289-1. A UGT can comprise a region that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% identical, or is 100% identical, to residues 83 to 92 of UGT94-289-1. A UGT can comprise a region that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% identical, or is 100% identical, to residues 179 to 198 of UGT94-289-1 (SEQ ID NO: 121).

In some embodiments, a host cell comprises a heterologous polynucleotide encoding a UGT, wherein the UGT comprises a region that: corresponds to residues 83 to 92 of wild-type UGT94-289-1 (SEQ ID NO: 121), wherein the region comprises an amino acid substitution relative to residues 83 to 92 of wild-type UGT94-289-1 (SEQ ID NO: 121); and/or corresponds to residues 179 to 198 of wild-type UGT94-289-1 (SEQ ID NO: 121), wherein the region comprises an amino acid substitution relative to residues 179 to 198 of wild-type UGT94-289-1 (SEQ ID NO: 121). It should be appreciated that the language “an amino acid substitution” is not limited to one amino acid substitution, but also encompasses embodiments including more than one amino acid substitution. In some embodiments, a host cell comprises a heterologous polynucleotide encoding a UGT, wherein the UGT comprises a region that: corresponds to residues 83 to 92 of wild-type UGT94-289-1 (SEQ ID NO: 121), wherein the region comprises no more than one amino acid substitution relative to residues 83 to 92 of wild-type UGT94-289-1 (SEQ ID NO: 121); and/or corresponds to residues 179 to 198 of wild-type UGT94-289-1 (SEQ ID NO: 121), wherein the region comprises no more than one amino acid substitution relative to residues 179 to 198 of wild-type UGT94-289-1 (SEQ ID NO: 121).

In some embodiments, the UGT comprises an amino acid substitution at an amino acid residue corresponding to the amino acid residue in wild-type UGT94-289-1 (SEQ ID NO: 121) selected from: H83, T84, T85, N86, P89, L92, Y179, S180, A181, G184, A185, V186, T187, K189, H191, K192, G194, E195, or A198.

In some embodiments, the residue corresponding to H83 is mutated to an amino acid comprising a polar uncharged R group or a nonpolar aromatic R group; the residue corresponding to T84 is mutated to an amino acid comprising a nonpolar aromatic R group; the residue corresponding to T85 is mutated to an amino acid comprising a nonpolar aliphatic R group, a positively charged R group, a polar uncharged R group, or a nonpolar aromatic R group; the residue corresponding to N86 is mutated to an amino acid comprising a nonpolar aliphatic R group, a polar uncharged R group, a negatively charged R group, a positively charged R group, or a nonpolar aromatic R group; the residue corresponding to P89 is mutated to an amino acid comprising a nonpolar aliphatic R group, or a polar uncharged R group; the residue corresponding to L92 is mutated to an amino acid comprising a positively charged R group; the residue corresponding to Y179 is mutated to an amino acid comprising a negatively charged R group, a nonpolar aromatic R group, a positively charged R group, or a nonpolar aliphatic R group; the residue corresponding to S180 is mutated to an amino acid comprising a nonpolar aliphatic R group; the residue corresponding to A181 is mutated to an amino acid comprising a positively charged R group or a polar uncharged R group; the residue corresponding to G184 is mutated to an amino acid comprising a nonpolar aliphatic R group, a polar uncharged R group, a negatively charged R group, a nonpolar aromatic R group, or a positively charged R group; the residue corresponding to A185 is mutated to an amino acid comprising a polar uncharged R group, a negatively charged R group, a nonpolar aliphatic R group, a positively charged R group, or a nonpolar aromatic R group; the residue corresponding to V186 is mutated to an amino acid comprising a nonpolar aliphatic R group, a polar uncharged R group, a negatively charged R group, a positively charged R group, or a nonpolar aromatic R group; the residue corresponding to T187 is mutated to an amino acid comprising a nonpolar aliphatic R group, a polar uncharged R group, a negatively charged R group, a positively charged R group, or a nonpolar aromatic R group; the residue corresponding to K189 is mutated to an amino acid comprising a nonpolar aliphatic R group, a polar uncharged R group, a negatively charged R group, a nonpolar aromatic R group, or a positively charged R group; the residue corresponding to H191 is mutated to an amino acid comprising a nonpolar aliphatic R group, a polar uncharged R group, a negatively charged R group, a positively charged R group, or a nonpolar aromatic R group; the residue corresponding to K192 is mutated to an amino acid comprising a polar uncharged R group or a nonpolar aromatic R group; the residue corresponding to G194 is mutated to an amino acid comprising a negatively charged R group, a nonpolar aliphatic R group, a polar uncharged R group, or a nonpolar aromatic R group; the residue corresponding to E195 is mutated to an amino acid comprising a nonpolar aliphatic R group, a positively charged R group, a polar uncharged R group, or a nonpolar aromatic R group; and/or the residue corresponding to A198 is mutated to an amino acid comprising a polar uncharged R group, a negatively charged R group, a nonpolar aromatic R group, a positively charged R group, or a nonpolar aliphatic R group.

In some embodiments, the UGT comprises an amino acid substitution at an amino acid residue corresponding to the amino acid residue in wild-type UGT94-289-1 (SEQ ID NO: 121) selected from: N143 and L374. In some embodiments, the residue corresponding to N143 is mutated to a negatively charged R group, a polar uncharged R group, or a nonpolar aliphatic R group. In some embodiments, the residue corresponding to L374 is mutated to a nonpolar aliphatic R group, a positively charged R group, a polar uncharged R group, or a nonpolar aromatic R group.

In some embodiments, a UGT does not comprise the sequence of SEQ ID NO: 121. In some embodiments, a UGT comprises less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 79%, less than 78%, less than 77%, less than 76%, less than 75%, less than 74%, less than 73%, less than 72%, less than 71%, less than 70%, less than 65%, less than 60%, less than 55%, less than 50%, less than 45%, less than 40%, less than 35%, or less than 30% identity to SEQ ID NO: 121.

### Cucurbitadienol Synthase (CDS) Enzymes

Aspects of the present disclosure provide cucurbitadienol synthase (CDS) enzymes, which may be useful, for example, in the production of a cucurbitadienol compound, such as 24-25 epoxy-cucurbitadienol or cucurbitadienol. CDSs are capable of catalyzing the formation of cucurbitadienol compounds, such as 24-25 epoxy-cucurbitadienol or cucurbitadienol from oxidosqualene (e.g., 2-3-oxidosqualene or 2,3; 22,23-diepoxysqualene).

In some embodiments, CDSs have a leucine at a residue corresponding to position 123 of SEQ ID NO: 256 that distinguishes them from other oxidosqualene cyclases, as discussed in Takase et al. Org. Biomol. Chem., 2015, 13, 7331-7336, which is incorporated by reference in its entirety.

CDSs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a nucleic acid or amino acid sequence in Table 9, to a sequence selected from SEQ ID NO: 184-263, 299, or 308, or to any other CDS sequence disclosed in this application or known in the art.

In some embodiments a CDS enzyme corresponds to AquAgaCDS16 (SEQ ID NO: 226), CSPI06G07180.1 (SEQ ID NO: 235), or A0A1S3CBF6 (SEQ ID NO: 232).

In some embodiments, a nucleic acid sequence encoding a CDS enzyme may be codon optimized for expression in a particular host cell, including S. cerevisiae. In some embodiments, a codon-optimized nucleic acid sequence encoding a CDS enzyme corresponds to SEQ ID NO: 186, 195 or 192.

In some embodiments, a CDS of the present disclosure is capable of using oxidosqualene (e.g., 2,3-oxidosqualene or 2,3; 22,23-diepoxysqualene) as a substrate. In some embodiments, a CDS of the present disclosure is capable of producing cucurbitadienol compounds (e.g., 24-25 epoxy-cucurbitadienol or cucurbitadienol). In some embodiments, a CDS of the present disclosure catalyzes the formation of cucurbitadienol compounds (e.g., 24-25 epoxy-cucurbitadienol or cucurbitadienol) from oxidosqualene (e.g., 2-3-oxidosqualene or 2,3; 22,23-diepoxysqualene).

It should be appreciated that activity of a CDS can be measured by any means known to one of ordinary skill in the art. In some embodiments, the activity of a CDS may be measured as the normalized peak area of cucurbitadienol produced. In some embodiments, this activity is measured in arbitrary units. In some embodiments, the activity, such as specific activity, of a CDS of the present disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control CDS.

It should be appreciated that one of ordinary skill in the art would be able to characterize a protein as a CDS enzyme based on structural and/or functional information associated with the protein. For example, in some embodiments, a protein can be characterized as a CDS enzyme based on its function, such as the ability to produce cucurbitadienol compounds (e.g., 24-25 epoxy-cucurbitadienol or cucurbitadienol) using oxidosqualene (e.g., 2,3-oxidosqualene or 2,3; 22,23-diepoxysqualene) as a substrate. In some embodiments, a protein can be characterized, at least in part, as a CDS enzyme based on the presence of a leucine residue at a position corresponding to position 123 of SEQ ID NO: 256.

In some embodiments, a host cell that comprises a heterologous polynucleotide encoding a CDS enzyme produces at least 10%, 20%, 30%, 40%, 50%, 60%, 70%, 80%, 90%, or 100% more cucurbitadienol compound compared to the same host cell that does not express the heterologous gene.

In other embodiments, a protein can be characterized as a CDS enzyme based on the percent identity between the protein and a known CDS enzyme. For example, the protein may be at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical, including all values in between, to any of the CDS sequences described in this application or the sequence of any other CDS enzyme. In other embodiments, a protein can be characterized as a CDS enzyme based on the presence of one or more domains in the protein that are associated with CDS enzymes. For example, in certain embodiments, a protein is characterized as a CDS enzyme based on the presence of a substrate channel and/or an active-site cavity characteristic of CDS enzymes known in the art. In some embodiments, the active site cavity comprises a residue that acts a gate to this channel, helping to exclude water from the cavity. In some embodiments, the active-site comprises a residue that acts a proton donor to open the epoxide of the substrate and catalyze the cyclization process.

In other embodiments, a protein can be characterized as a CDS enzyme based on a comparison of the three-dimensional structure of the protein compared to the three-dimensional structure of a known CDS enzyme. It should be appreciated that a CDS enzyme can be a synthetic protein.

### C11 Hydroxylase Enzymes

Aspects of the present disclosure provide C11 hydroxylase enzymes, which may be useful, for example, in the production of mogrol.

A C11 hydroxylase of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a C11 hydroxylase sequence (e.g., nucleic acid or amino acid sequence) in Tables 10 and 11, with a sequence set forth as SEQ ID NO: 264-265, 280-281, 296, or 305, or to any C11 hydroxylase sequence disclosed in this application or known in the art.

In some embodiments, a C11 hydroxylase of the present disclosure is capable of oxidizing mogrol precursors (e.g., cucurbitadienol, 11-hydroxycucurbitadienol, 24,25-dihydroxy-cucurbitadienol, and/or 24,25-epoxy-cucurbitadienol). In some embodiments, a C11 hydroxylase of the present disclosure catalyzes the formation of mogrol.

It should be appreciated that activity, such as specific activity, of a C11 hydroxylase can be determined by any means known to one of ordinary skill in the art. In some embodiments, activity (e.g., specific activity) of a C11 hydroxylase may be measured as the concentration of a mogrol precursor produced or mogrol produced per unit of enzyme per unit time. In some embodiments, a C11 hydroxylase of the present disclosure has an activity (e.g., specific activity) of at least 0.0001-0.001 µmol/min/mg, at least 0.001-0.01 µmol/min/mg, at least 0.01-0.1 µmol/min/mg, or at least 0.1-1 µmol/min/mg, including all values in between.

In some embodiments, the activity, such as specific activity, of a C11 hydroxylase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control C11 hydroxylase.

### Cytochrome P450 Reductase Enzymes

Aspects of the present disclosure provide cytochrome P450 reductase enzymes, which may be useful, for example, in the production of mogrol. Cytochrome P450 reductase is also referred to as NADPH:ferrihemoprotein oxidoreductase, NADPH:hemoprotein oxidoreductase, NADPH:P450 oxidoreductase, P450 reductase, POR, CPR, and CYPOR. These reductases can promote C11 hydroxylase activity by catalyzing electron transfer from NADPH to a C11 hydroxylase.

Cytochrome P450 reductases of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a cytochrome P450 reductase sequence (e.g., nucleic acid or amino acid sequence) in Tables 10 and 11, with a sequence set forth as SEQ ID NO: 266-267, 282-283, 297-298, or 306-307, or to any cytochrome p450 reductase disclosed in this application or known in the art.

In some embodiments, a cytochrome P450 reductase of the present disclosure is capable of promoting oxidation of a mogrol precursor (e.g., cucurbitadienol, 11-hydroxycucurbitadienol, 24,25-dihydroxy-cucurbitadienol, and/or 24,25-epoxy-cucurbitadienol). In some embodiments, a P450 reductase of the present disclosure catalyzes the formation of a mogrol precursor or mogrol.

It should be appreciated that activity (e.g., specific activity) of a cytochrome P450 reductase can be measured by any means known to one of ordinary skill in the art. In some embodiments, activity (e.g., specific activity) of a recombinant cytochrome P450 reductase may be measured as the concentration of a mogrol precursor produced or mogrol produced per unit enzyme per unit time in the presence of a C11 hydroxylase. In some embodiments, a cytochrome P450 reductase of the present disclosure has a activity (e.g., specific activity) of at least 0.0001-0.001 µmol/min/mg, at least 0.001-0.01 µmol/min/mg, at least 0.01-0.1 µmol/min/mg, or at least 0.1-1 µmol/min/mg, including all values in between.

In some embodiments, the activity (e.g., specific activity) of a cytochrome P450 reductase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control cytochrome P450 reductase.

### Epoxide Hydrolase Enzymes (EPHs)

Aspects of the present disclosure provide epoxide hydrolase enzymes (EPHs), which may be useful, for example, in the conversion of 24-25 epoxy-cucurbitadienol to 24-25 dihydroxy-cucurbitadienol or in the conversion of 11-hydroxy-24,25-epoxycucurbitadienol to mogrol. EPHs are capable of converting an epoxide into two hydroxyls.

EPHs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a EPH sequence (e.g., nucleic acid or amino acid sequence) in Tables 10 and 11, with a sequence set forth as SEQ ID NO: 268-276, 284-292, 300-301 or 309-310, or to any EPH sequence disclosed in this application or known in the art.

In some embodiments, a recombinant EPH of the present disclosure is capable of promoting hydrolysis of an epoxide in a cucurbitadienol compound (e.g., hydrolysis of the epoxide in 24-25 epoxy-cucurbitadienol). In some embodiments, an EPH of the present disclosure catalyzes the formation of a mogrol precursor (e.g., 24-25 dihydroxy-cucurbitadienol).

It should be appreciated that activity (e.g., specific activity) of an EPH can be measured by any means known to one of ordinary skill in the art. In some embodiments, activity (e.g., specific activity) of an EPH may be measured as the concentration of a mogrol precursor (e.g., 24-25 dihydroxy-cucurbitadienol) or mogrol produced. In some embodiments, a recombinant EPH of the present disclosure will allow production of at least 1-100 µg/L, at least 100-1000 µg/L, at least 1-100 mg/L, at least 100-1000 mg/L, at least 1-10 g/L or at least 10-100 g/L, including all values in between.

In some embodiments, the activity (e.g., specific activity) of an EPH is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control EPH.

### Squalene Epoxidases Enzymes (SQEs)

Aspects of the present disclosure provide squalene epoxidases (SQEs), which are capable of oxidizing a squalene (e.g., squalene or 2-3-oxidosqualene) to produce a squalene epoxide (e.g., 2-3-oxidosqualene or 2-3, 22-23-diepoxysqualene). SQEs may also be referred to as squalene monooxygenases.

SQEs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a SQE sequence (e.g., nucleic acid or amino acid sequence) in Tables 10 and 11, with a sequence set forth as SEQ ID NO: 277-279, 293-295, 303 or 312, or to any SQE sequence disclosed in this application or known in the art.

In some embodiments, an SQE of the present disclosure is capable of promoting formation of an epoxide in a squalene compound (e.g., epoxidation of squalene or 2,3-oxidosqualene). In some embodiments, an SQE of the present disclosure catalyzes the formation of a mogrol precursor (e.g., 2-3-oxidosqualene or 2-3, 22-23-diepoxysqualene). Activity, such as specific activity, of a recombinant SQE may be measured as the concentration of a mogrol precursor (e.g., 2-3-oxidosqualene or 2-3, 22-23-diepoxysqualene) produced per unit of enzyme per unit of time. In some embodiments, an SQE of the present disclosure has an activity, such as specific activity, of at least 0.0000001 µmol/min/mg (e.g., at least 0.000001 µmol/min/mg, at least 0.00001 µmol/min/mg, at least 0.0001 µmol/min/mg, at least 0.001 µmol/min/mg, at least 0.01 µmol/min/mg, at least 0.1 µmol/min/mg, at least 1 µmol/min/mg, at least 10 µmol/min/mg, or at least 100 µmol/min/mg, including all values in between).

In some embodiments, the activity, such as specific activity, of a SQE is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control SQE.

### Variants

Aspects of the disclosure relate to polynucleotides encoding any of the recombinant polypeptides described, such as CDS, UGT, C11 hydroxylase, cytochrome P450 reductase, and EPH, and SQE enzymes. Variants of polynucleotide or amino acid sequences described in this application are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, while in other embodiments, sequence identity is determined over a region of a sequence.

Identity can also refer to the degree of sequence relatedness between two sequences as determined by the number of matches between strings of two or more residues (e.g., nucleic acid or amino acid residues). Identity measures the percent of identical matches between the smaller of two or more sequences with gap alignments (if any) addressed by a particular mathematical model, algorithms, or computer program.

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the protein molecules of the invention. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T.F. & Waterman, M.S. (1981) “Identification of common molecular subsequences.” J. Mol. Biol. 147:195-197). A general global alignment technique which may be used, for example, is the Needleman-Wunsch algorithm (Needleman, S.B. & Wunsch, C.D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct 11;7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T.F. & Waterman, M.S. (1981) “Identification of common molecular subsequences.” J. Mol. Biol. 147:195-197) or the Needleman-Wunsch algorithm (Needleman, S.B. & Wunsch, C.D. (1970) “A general method applicable to the search for similarities in the amino acid sequences of two proteins.” J. Mol. Biol. 48:443-453).

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA).

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct 11;7:539).

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art.

Variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant (e.g., CDS, UGT, C11 hydroxylase, cytochrome P450 reductase, EPH, or SQE variant) comprises a domain that shares a secondary structure (e.g., alpha helix, beta sheet) with a reference polypeptide (e.g., a reference CDS, UGT, C11 hydroxylase, cytochrome P450 reductase, EPH, or SQE). In some embodiments, a polypeptide variant (e.g., CDS, UGT, C11 hydroxylase, cytochrome P450 reductase, EPH, or SQE variant) shares a tertiary structure with a reference polypeptide (e.g., a reference CDS, UGT, C11 hydroxylase, cytochrome P450 reductase, EPH, or SQE). As a non-limiting example, a variant polypeptide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets, or have the same tertiary structure as a reference polypeptide. For example, a loop may be located between a beta sheet and an alpha helix, between two alpha helices, or between two beta sheets. Homology modeling may be used to compare two or more tertiary structures.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing tools, or by insertions, such as insertion of a tag (e.g., a HIS tag or a GFP tag). Mutations can include, for example, substitutions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F.M. Ausubel, et al., eds., John Wiley & Sons, Inc., New York, 2010.

In some embodiments, methods for producing variants include circular permutation (Yu and Lutz, Trends Biotechnol. 2011 Jan;29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 Jan;29(1): 18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr 1;21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of the recombinant CDSs, UGTs, C11 hydroxylases, cytochrome P450 reductases, EPHs, and squalene epoxidases disclosed in this application are also encompassed by the present disclosure. For example, functional variants may bind one or more of the same substrates (e.g., mogrol, mogroside, or precursors thereof) or produce one or more of the same products (e.g., mogrol, mogroside, or precursors thereof). Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 Jul;28(3):405-20) may be used to identify polypeptides with a particular domain. For example, among oxidosqualene cyclases, additional CDS enzymes may be identified in some instances by searching for polypeptides with a leucine residue corresponding to position 123 of SEQ ID NO: 256. This leucine residue has been implicated in determining the product specificity of the CDS enzyme; mutation of this residue can, for instance, result in cycloartenol or parkeol as a product (Takase et al., Org Biomol Chem. 2015 Jul 13(26):7331-6).

Additional UGT enzymes may be identified, for example, by searching for polypeptides with a UDPGT domain (PROSITE accession number PS00375).

Homology modeling may also be used to identify amino acid residues that are amenable to mutation without affecting function. A non-limiting example of such a method may include use of position-specific scoring matrix (PSSM) and an energy minimization protocol. See, e.g., Stormo et al., Nucleic Acids Res. 1982 May 11;10(9):2997-3011.

PSSM may be paired with calculation of a Rosetta energy function, which determines the difference between the wild-type and the single-point mutant. Without being bound by a particular theory, potentially stabilizing mutations are desirable for protein engineering (e.g., production of functional homologs). In some embodiments, a potentially stabilizing mutation has a ΔΔG_{calc} value of less than -0.1 (e.g., less than -0.2, less than -0.3, less than -0.35, less than -0.4, less than -0.45, less than -0.5, less than -0.55, less than -0.6, less than -0.65, less than -0.7, less than -0.75, less than -0.8, less than -0.85, less than -0.9, less than -0.95, or less than -1.0) Rosetta energy units (R.e.u.). See, e.g., Goldenzweig et al., Mol Cell. 2016 Jul 21;63(2):337-346. doi: 10.1016/j.molcel.2016.06.012.

In some embodiments, a CDS, UGT, C11 hydroxylase, cytochrome P450 reductase, EPH, or SQE coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference coding sequence. In some embodiments, the CDS, UGT, C11 hydroxylase, cytochrome P450 reductase, EPH, or SQE coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a recombinant CDS, UGT, C11 hydroxylase, cytochrome P450 reductase, EPH, or SQE sequence alter the amino acid sequence of the polypeptide relative to the amino acid sequence of a reference polypeptide. In some embodiments, the one or more mutations alter the amino acid sequence of the recombinant polypeptide relative to the amino acid sequence of a reference polypeptide and alter (enhance or reduce) an activity of the polypeptide relative to the reference polypeptide.

The activity, including specific activity, of any of the recombinant polypeptides described in this application may be measured using methods known in the art. As a non-limiting example, a recombinant polypeptide’s activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 2). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 2.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 2

Non-limiting Examples of conservative amino acid substitutions

| | | |
|---|---|---|
| Original Residue | R Group Type | Conservative Amino Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg. Tyr, Trp |
| Ile | nonpolar aliphatic R group | Leu, Met, Val |
| Leu | nonpolar aliphatic R group | Ile. Met, Val |
| Lys | positively charged R group | Arg, His |
| Met | nonpolar aliphatic R group | Ile. Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide (e.g., UGT, CDS, P450, cytochrome P450 reductase, EPH, or squalene epoxidase).

### Expression of Nucleic Acids in Host Cells

Aspects of the present disclosure relate to the recombinant expression of genes encoding enzymes, functional modifications and variants thereof, as well as uses relating thereto. For example, the methods described in this application may be used to produce mogrol precursors, mogrol and/or mogrosides.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 Jul; 13(7): 563-567. A heterologous polynucleotide may comprise a wild-type sequence or a mutant sequence as compared with a reference polynucleotide sequence.

A nucleic acid encoding any of the recombinant polypeptides, such as CDSs, UGTs, C11 hydroxylases, cytochrome P450 reductases, EPHs, or SQEs, described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

In some embodiments, a vector replicates autonomously in the cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell, such as a yeast cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, the nucleic acid sequence of a gene described in this application is codon-optimized. Codon optimization may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined or linked if induction of a promoter in the 5′ regulatory sequence permits the coding sequence to be transcribed and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1,TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Plslcon, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. Non-limiting examples of inducible promoters include chemically-regulated promoters and physically-regulated promoters. For chemically-regulated promoters, the transcriptional activity can be regulated by one or more compounds, such as alcohol, tetracycline, galactose, a steroid, a metal, or other compounds. For physically-regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1,TPI1, HXT3, HXT7, ACT1, ADH1, ADH2, ENO2, and SOD1.

Other inducible promoters or constitutive promoters known to one of ordinary skill in the art are also contemplated.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed in this application may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a host cell is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

In some embodiments, introduction of a polynucleotide, such as a polynucleotide encoding a recombinant polypeptide, into a host cell results in genomic integration of the polynucleotide. In some embodiments, a host cell comprises at least 1 copy, at least 2 copies, at least 3 copies, at least 4 copies, at least 5 copies, at least 6 copies, at least 7 copies, at least 8 copies, at least 9 copies, at least 10 copies, at least 11 copies, at least 12 copies, at least 13 copies, at least 14 copies, at least 15 copies, at least 16 copies, at least 17 copies, at least 18 copies, at least 19 copies, at least 20 copies, at least 21 copies, at least 22 copies, at least 23 copies, at least 24 copies, at least 25 copies, at least 26 copies, at least 27 copies, at least 28 copies, at least 29 copies, at least 30 copies, at least 31 copies, at least 32 copies, at least 33 copies, at least 34 copies, at least 35 copies, at least 36 copies, at least 37 copies, at least 38 copies, at least 39 copies, at least 40 copies, at least 41 copies, at least 42 copies, at least 43 copies, at least 44 copies, at least 45 copies, at least 46 copies, at least 47 copies, at least 48 copies, at least 49 copies, at least 50 copies, at least 60 copies, at least 70 copies, at least 80 copies, at least 90 copies, at least 100 copies, or more, including any values in between, of a polynucleotide sequence, such as a polynucleotide sequence encoding any of the recombinant polypeptides described in this application, in its genome.

### Host Cells

Any of the proteins or enzymes of the disclosure may be expressed in a host cell. As used in this application, the term “host cell” refers to a cell that can be used to express a polynucleotide, such as a polynucleotide that encodes an enzyme used in production of mogrol, mogrosides, and precursors thereof.

Any suitable host cell may be used to produce any of the recombinant polypeptides, including CDSs, UGTs, C11 hydroxylases, cytochrome P450 reductases, EPHs, and SQEs disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranifaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolyticaIn some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. Reinhardtii) and Phormidium ( P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora,Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host cell is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88 , C. lituseburense, C. saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. carotovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell is an industrial Zymomonas species (e.g., Z. mobilis, Z. lipolytica).

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), and hybridoma cell lines.

The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells.

The host cell may comprise genetic modifications relative to a wild-type counterpart. As a non-limiting example, a host cell (e.g., S. cerevisiae) may be modified to reduce or inactivate one or more of the following genes: hydroxymethylglutaryl-CoA (HMG-CoA) reductase (HMG1), acetyl-CoA C-acetyltransferase (acetoacetyl-CoA thiolase) (ERG10), 3-hydroxy-3-methylglutaryl-CoA (HMG-CoA) synthase (ERG13), farnesyl-diphosphate farnesyl transferase (squalene synthase) (ERG9), may be modified to overexpress squalene epoxidase (ERG1), or may be modified to downregulate lanosterol synthase (ERG7). In some embodiments, a host cell is modified to reduce or eliminate expression of one or more transporter genes, such as PDR1 or PDR3, and/or the glucanase gene EXG1.

In some embodiments, a host cell is modified to reduce or inactivate at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 genes.

In some embodiments, a host cell is modified to reduce or inactivate 1, 2, 3, 4, 5, 6, 7, 8, 9, 10, 11, 12, 13, 14, 15, 16, 17, 18, 19, or 20 genes.

Reduction of gene expression and/or gene inactivation may be achieved through any suitable method, including but not limited to deletion of the gene, introduction of a point mutation into the gene, truncation of the gene, introduction of an insertion into the gene, introduction of a tag or fusion into the gene, or selective editing of the gene. For example, polymerase chain reaction (PCR)-based methods may be used (see, e.g., Gardner et al., Methods Mol Biol. 2014;1205:45-78) or well-known gene-editing techniques may be used. As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104).

A vector encoding any of the recombinant polypeptides described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006;313:107-20, which is incorporated by reference in its entirety. Host cells may be cultured under any suitable conditions as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermentor is used to culture the cell. Thus, in some embodiments, the cells are used in fermentation. As used in this application, the terms “bioreactor” and “fermentor” are interchangeably used and refer to an enclosure, or partial enclosure, in which a biological, biochemical and/or chemical reaction takes place, involving a living organism, part of a living organism, or purified enzymes. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermentors, bioreactors agitated by rotating mixing devices, chemostats, bioreactors agitated by shaking devices, airlift fermentors, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermentors, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or nonporous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermentor includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction byproducts), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., mogrol precursor, mogrol, mogroside precursor, or mogroside) may display some differences from the substrate (e.g., mogrol precursor, mogrol, mogroside precursor, or mogroside) in terms of solubility, toxicity, cellular accumulation and secretion and in some embodiments can have different fermentation kinetics.

The methods described in this application encompass production of the mogrol precursors (e.g., squalene, 2,3-oxidosqualene, or 24-25 epoxy-cucurbitadienol), mogrol, or mogrosides (e.g., MIA1, MIE1, MIIA1, MIIA2, MIIIA1, MIIE1, MIII, siamenoside I, mogroside IV, isomogroside IV, MIIIE, and mogroside V) using a recombinant cell, cell lysate or isolated recombinant polypeptides (e.g., CDS, UGT, C11 hydroxylase, cytochrome P450 reductase, EPH, and squalene epoxidase).

Mogrol precursors (e.g., squalene, 2,3-oxidosqualene, or 24-25 epoxy-cucurbitadienol), mogrol, mogrosides (e.g., MIA1, MIE, MIIA1, MIIA2, MIIIA1, MIIE1, MIII, siamenoside I, mogroside IV, isomogroside IV, MIIIE, and mogroside V) produced by any of the recombinant cells disclosed in this application may be identified and extracted using any method known in the art. Mass spectrometry (e.g., LC-MS, GC-MS) is a non-limiting example of a method for identification and may be used to help extract a compound of interest.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1. Identification and Functional Characterization of Putative UGT Enzymes With Primary Glycosylation Activity

This Example describes the screening of a UGT library to identify UGTs capable of converting mogrol and mogroside precursors into glycosylated mogrosides. Specifically, the library aimed to identify UGTs with primary glycoslyation activity that glycosylate at the C3 and C24 hydroxyl groups of mogrol to yield mogrosides with different glucose units. The library included approximately 2152 putative UGTs that were obtained from both plant and non-plant sources. The entire library was screened for primary glycosylation activity.

To test the UGT library for primary glycosylation activity, an in vivo plate assay combined with analysis by LC-MS was utilized. Plasmids carrying individual UGTs were transformed and integrated into the chromosome of a S. cerevisiae chassis strain that produces a mogrol precursor. A strain expressing a UGT that forms single glycosylation products was used as a positive control, and a strain expressing GFP was used as a negative control.

Single colonies resulting from transformation were grown as pre-cultures containing culturing media in a shaking incubator at 30° C. for 48 hours at 1000 rpm. After 48 hours, pre-cultures were transferred into production media and grown in a shaking incubator at 30° C. for 96 hours at 1000 rpm. After 96 hours, cultures were extracted with an organic solvent and product formation was tested by LC-MS analysis to evaluate mogroside production. A thermo QQQ TSQ-Quantiva ESI with a LX4 multiplexed columns setup was used. A C18 column (Accucore 100 ×2.1 mm) with a 0.1% formic acid in water running buffer and acetonitrile ramp were used for separation in negative mode using SIM.

Initially, a short analytical run was performed to identify product species based on mass. Based on this screen, 250 candidate UGTs were identified that demonstrated primary glycosylation activity.

A subset of candidate UGTs was selected based on their mogrol conversion activities. These UGTs were subjected to follow-up analysis with a longer analytical run to characterize the mogroside products as MIA1 (C24 glycosylation) MIE1 (C3 glycosylation) or MIIE (C24 and C3 glycosylation).

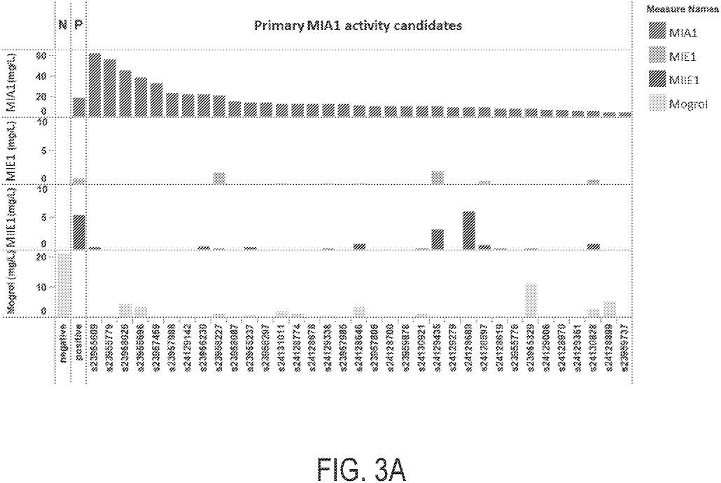

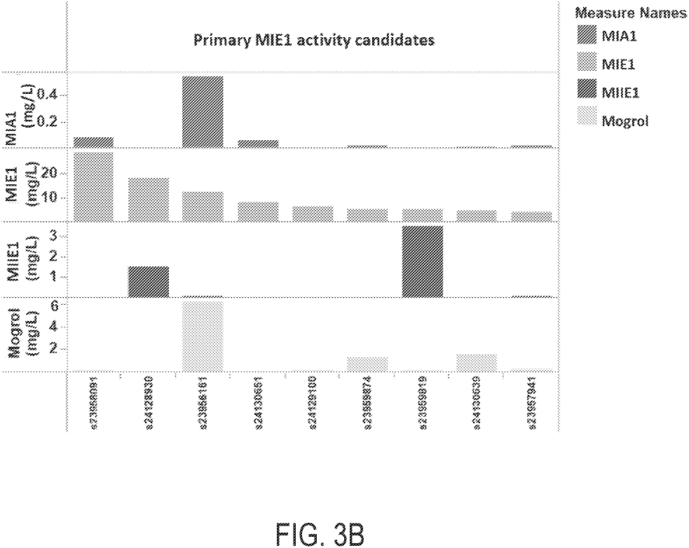

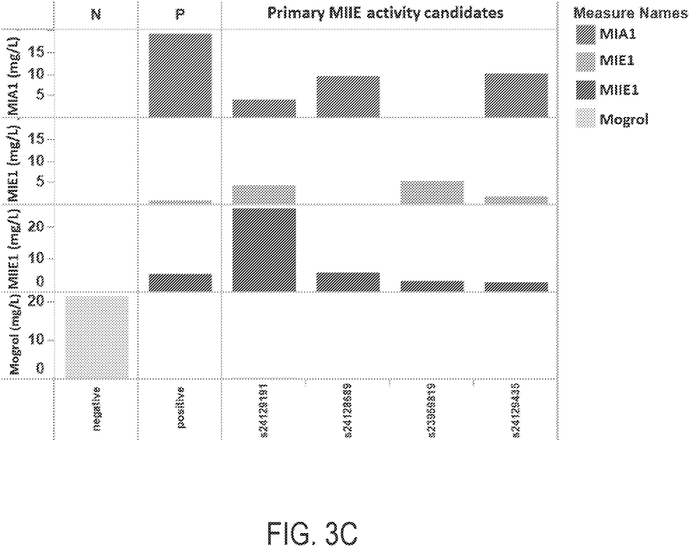

FIGS. 2 and 3 demonstrate results from the primary glycosylation UGT library screen. FIG. 2 shows results from the longer analytical run carried out on the subset of candidate UGTs described above to characterize the mogroside products identified. FIG. 3 depicts the UGT candidates from FIG. 2 that demonstrated considerable glycosylation activity. FIG. 3A demonstrates candidates with MIA1 (C24 glycosylation) activity, FIG. 3B demonstrates candidates with MIE1 (C3 glycosylation) and FIG. 3C demonstrates candidate with MIIE1 (C3 and C24 glycosylation) activity. These data are also summarized in Table 3. As a non-limiting example, Table 3 shows that Sample ID 24128597 comprising UGT ID/UGT protein m3954168 (SEQ ID NO: 78) demonstrated UGT primary glycosylation activity of converting mogrol to mogrosides MIA1, MIE1, and MIIE1. This sample produced 9.44 mg/L MIA1; 0.62 mg/L MIE1; 0.78 mg/L MIIE1; and 0.13 mg/L Mogrol.

TABLE 3

Primary glycosylation by Candidate UGTs

| | Sample ID | UGT ID/ UGT protein SEQ ID NO: | Type | MIA1 (mg/L) | MIE1 (mg/L) | MIIE1 (mg/L) | Mogrol (mg/L) |
|---|---|---|---|---|---|---|---|
| MIA1 | 24128597 | m3954168 SEQ ID NO: 78 | Library | 9.44 | 0.62 | 0.78 | 0.13 |
| | 24128689 | m3954684 SEQ ID NO: 94 | Library | 9.64 | 0.08 | 5.88 | 0.09 |
| | 23958087 | m3710896 | Library | 14.9 | 0 | 0.02 | 0.13 |
| | 23956230 | m3953983 SEQ ID NO: 76 | Library | 22.08 | 0.04 | 0.52 | 0.1 |
| | 23959878 | m3954531 SEQ ID NO: 88 | Library | 10.59 | 0.01 | 0 | 0.11 |
| | 24128970 | m3954606 SEQ ID NO: 89 | Library | 6.67 | 0 | 0 | 0.07 |
| | 24129351 | m3954350 SEQ ID NO: 86 | Library | 6.25 | 0 | 0.01 | 0.08 |
| | 24128678 | m3954761 SEQ ID NO: 97 | Library | 12.59 | 0 | 0 | 0.06 |
| | 24128774 | m3954639 SEQ ID NO: 92 | Library | 13.04 | 0.08 | 0.07 | 1.08 |
| | 23957806 | m3710904 SEQ ID NO: 66 | Library | 11.08 | 0.01 | 0 | 0.13 |
| | 24129435 | m3954307 SEQ ID NO: 84 | Library | 10.32 | 1.96 | 3.02 | 0 |
| | 23955237 | m3953464 SEQ ID NO: 69 | Library | 14.42 | 0 | 0.35 | 0.62 |
| | 24129338 | m3954267 | Library | 12.38 | 0.12 | 0.1 | 0.32 |
| | 24129142 | m3953871 SEQ ID NO: 73 | Library | 22.22 | 0 | 0 | 0.08 |
| | 23957988 | m3954751 SEQ ID NO: 96 | Library | 23.37 | 0.01 | 0.04 | 0.21 |
| | 24128646 | m3954669 SEQ ID NO: 93 | Library | 12.35 | 0.17 | 0.86 | 3.5 |
| | 23957459 | m3955387 SEQ ID NO: 101 | Library | 33.56 | 0 | 0.04 | 0.21 |
| | 23958227 | m3953189 SEQ 67NO: | Library | 21.71 | 1.82 | 0.24 | 1.32 |
| | 23955779 | m3954308 SEQ ID NO: 85 | Library | 56.79 | 0 | 0 | 0.34 |
| | 23958026 | m3954637 | Library | 45.81 | 0.03 | 0.05 | 4.46 |
| | | SEQ ID NO: 91 | | | | | |
| | 24129006 | m3710903 SEQ ID NO: 65 | Library | 7.18 | 0 | 0 | 0.36 |
| | 24130828 | m3953734 SEQ ID NO: 72 | Library | 5.33 | 0.75 | 0.95 | 2.66 |
| | 24128619 | m3953944 SEQ ID NO: 75 | Library | 8.05 | 0.04 | 0.1 | 0.32 |
| | 23955329 | m3953567 SEQ ID NO: 70 | Library | 7.99 | 0.02 | 0.18 | 11.22 |
| | 23955609 | m3953702 SEQ ID NO: 71 | Library | 62.84 | 0.02 | 0.36 | 0.43 |
| | 23958297 | m3953262 SEQ ID NO: 68 | Library | 13.99 | 0 | 0 | 0.12 |
| | 23959737 | m3710903 SEQ ID NO: 65 | Library | 4.82 | 0 | 0 | 0.4 |
| | 24130921 | m3954997 SEQ ID NO: 98 | Library | 10.54 | 0.02 | 0.17 | 1.09 |
| | 23957985 | m3954742 SEQ ID NO: 95 | Library | 12.38 | 0.02 | 0.03 | 0.18 |
| | 24129279 | m3954259 SEQ ID NO: 81 | Library | 9.92 | 0 | 0 | 0.27 |
| | 23955696 | m3955013 SEQ ID NO: 99 | Library | 38.75 | 0.01 | 0 | 3.42 |
| | 24128700 | m3954742 SEQ ID NO: 95 | Library | 10.66 | 0 | 0 | 0.21 |
| | 24128899 | m3954610 SEQ ID NO: 90 | Library | 5.17 | 0 | 0 | 5.1 |
| | 24131011 | m3955034 | Library | 13.4 | 0.15 | 0.04 | 2.1 |
| | | SEQ ID NO: 100 | | | | | |
| | 23955776 | m3954283 SEQ ID NO: 83 | Library | 8.04 | 0 | 0 | 0.07 |
| MIE1 | 23957941 | m3955517 SEQ ID NO: 102 | Library | 0.02 | 4.16 | 0.05 | 0.19 |
| | 24128930 | m3710890 SEQ ID NO: 63 | Library | 0 | 17.96 | 1.53 | 0.01 |
| | 23958091 | m3710885 SEQ ID NO: 62 | Library | 0.08 | 28.79 | 0.03 | 0.06 |
| | 23959874 | m3954446 SEQ ID NO: 87 | Library | 0.02 | 5.47 | 0.02 | 1.29 |
| | 24130651 | m3954254 | Library | 0.06 | 8.34 | 0.02 | 0.01 |
| | 24129100 | m3955521 SEQ ID NO: 103 | Library | 0 | 6.4 | 0 | 0.17 |
| | 24130639 | m3954239 SEQ ID NO: 79 | Library | 0.01 | 4.96 | 0 | 1.57 |
| | 23956161 | m3953937 SEQ ID NO: 74 | Library | 0.55 | 12.61 | 0.09 | 6.28 |
| MIIE1 | 24128689 | m3954684 SEQ ID NO: 94 | Library | 9.64 | 0.08 | 5.88 | 0.09 |
| | 24129435 | m3954307 SEQ ID NO: 84 | Library | 10.32 | 1.96 | 3.02 | 0 |
| | 23959819 | m3710890 SEQ ID NO: 63 | Library | 0 | 5.44 | 3.57 | 0.17 |
| | 24129191 | m3954005 SEQ ID NO: 77 | Library | 4.2 | 4.56 | 25.68 | 0.22 |
| Control | 23955762 | m3778973 SEQ ID NO: 117 | Negative Control | 0.02 | 0.01 | 0 | 24.85 |
| | 23956146 | m3778973 SEQ ID NO: 117 | Negative Control | 0.07 | 0 | 0 | 17.12 |
| | 24129380 | m3778975 | Positive control | 11.48 | 0.63 | 1.29 | 0.08 |
| | 24129092 | m3778975 | Positive control | 11.87 | 0.65 | 1.45 | 0.21 |

### Example 2. Identification and Functional Characterization of Putative UGT Enzymes With Secondary Glycosylation Activity

This Example describes the identification of UGTs with secondary glycosylation activity. A library of approximately 880 candidate UGTs was generated. The candidate UGTs within this library corresponded to candidate UGTs within the library described in Example 1 that were obtained from plant sources.

To test the UGT library for secondary glycosylation activity, an in vivo plate assay combined with analysis by LC-MS was utilized. Plasmids carrying individual UGTs were transformed and integrated into the chromosome of a chassis strain that produces primary mogrosides. A strain expressing a UGT that forms double glycosylated products was used as a positive control, and a strain expressing GFP was used as a negative control.

Single colonies resulting from transformation were grown as pre-cultures containing culturing media in a shaking incubator at 30° C. for 48 hours at 1000 rpm. After 48 hours, pre-cultures were transferred into production media and grown in a shaking incubator at 30° C. for 96 hours at 1000 rpm. After 96 hours, cultures were extracted with an organic solvent and product formation was tested by LC-MS analysis to evaluate mogroside production. A thermo QQQ TSQ-Quantiva ESI with a LX4 multiplexed columns setup was used. A C18 column (Accucore 100 ×2.1 mm) with a 0.1% formic acid in water running buffer and acetonitrile ramp were used for separation in negative mode using SIM. Initially, analysis was performed to identify product species based on mass. Based on this screen, two candidate UGTs (sample s23953261, expressing a candidate UGT corresponding to SEQ ID NO: 104 and sample s23953509, expressing a candidate UGT corresponding to SEQ ID NO: 105) were identified that demonstrated secondary glycosylation activity, including production of MIIIs, MIVs and MVs.

Follow-up analysis with a longer analytical run was performed on these two candidate UGTs to characterize the mogroside products. Surprisingly, sample s23953509 (expressing SEQ ID NO: 105) demonstrated production of MIVA but not other types of MIVs and also did not produce substantial amounts of MV products, indicating very specific 1-6 glycosylation activity. Sample s23953261 (expressing SEQ ID NO: 104) demonstrated production of MIII and MIV products that differed from the standards.

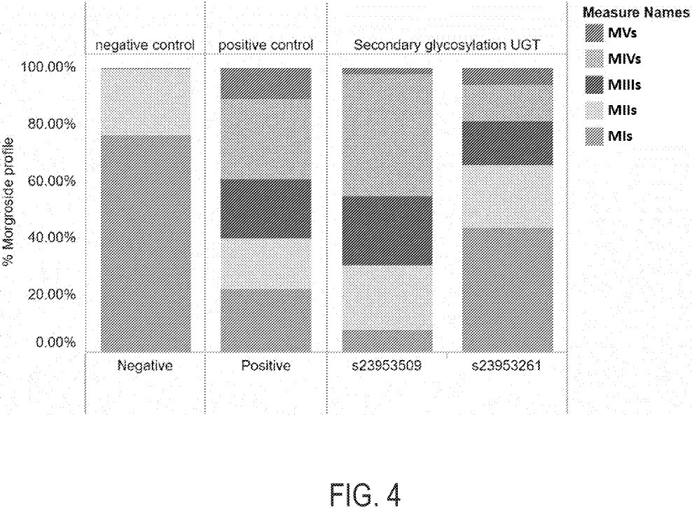

FIG. 4 and Table 4 depict mogroside production profiles from the two candidate UGTs identified. NPA indicates normalized peak area, which is calculated as the area under the curve based on LC/MS data and normalized to an internal analytical standard. The amount of mogrol (M), MIs, MIIs, MIIIs, MIVs, and MVs produced by strains carrying each of the 2 candidate UGTs were compared to the positive control strain. The fraction of MIs, MIIs, MIIIs, MIVs, and MVs corresponds to the amount of each relative to the total amount of product produced. As a non-limiting example, in Table 4, Sample ID s23953261 comprising UGT ID/UGT protein m3953578 (SEQ ID NO: 104) demonstrated secondary glycosylation activity of catalyzing the addition of a glycosyl group to a position on a compound that already comprises a glycosyl group. Sample ID s23953261 produced MIIIs, MIVs, and MVs; in particular, this sample produced: M (NPA): 0; MI (NPA): 1.76; MII (NPA): 0.91; MIII (NPA): 0.62; MIV (NPA): 0.52; and MV (NPA): 0.23.

TABLE 4

Secondary Glycosylation Activity Data from UGT Library

| Sample ID | UGT ID/ UGT protein SEQ ID NO | Type | M (NPA) | MI (NPA) | MII (NPA) | MIII (NPA) | MIV (NPA) | MV (NPA) |
|---|---|---|---|---|---|---|---|---|
| s23953261 | m3953578 SEQ ID NO: 104 | Library | 0 | 1.76 | 0.91 | 0.62 | 0.52 | 0.23 |
| s23953509 | m3953963 SEQ ID NO: 105 | Library | 0 | 0.64 | 1.86 | 2.05 | 3.55 | 0.17 |
| s24129705 | m3778973 SEQ ID NO: 117 | Negative Control | 0.01 | 6.44 | 0.21 | 0 | 0 | 0 |
| s24129477 | m3778974 | Positive Control | 0 | 0.69 | 0.11 | 0.19 | 0.39 | 0.09 |

### Example 3. Further Identification and Functional Characterization of Candidate UGT Enzymes With Secondary Glycosylation Activity

This Example describes the design and screening of an additional UGT library to identify additional UGTs with secondary glycoslyation activity. The UGT library consisted of approximately 163 candidate UGTs.

To test the UGT library for secondary glycosylation activity, an in vivo plate assay combined with analysis by LC-MS was utilized. Plasmids carrying UGTs were transformed into a chassis strain that expressed all of the enzymes required to make mogrol as well as two primary UGTs for production of primary mogrosides. The UGT library was screened in biological duplicates and evaluated initially using the same short analytical method as described in Examples 1 and 2.

Selected candidate UGTs were chosen for additional analysis using the longer analytical method, as described in Examples 1 and 2, to further characterize their activity through the identification of mogroside products. These candidate UGTs were run in four biological replicates and results were compared with a negative control expressing GFP, and a positive control secondary UGT.

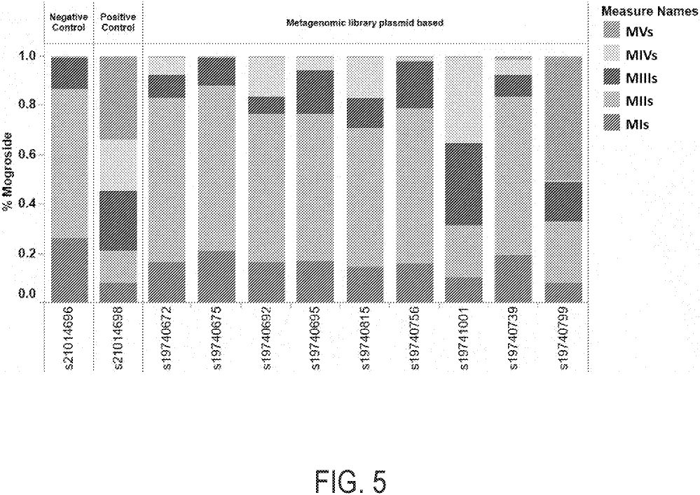

Based on this screen, 9 candidate UGTs were identified that demonstrated secondary glycosylation activity and could produce mogroside products, including MIIIs, MIVs, and MVs. The percentages of mogrol, MIs, MIIs, MIIIs, MIVs, and MVs produced by these strains are shown in Table 5 and FIG. 5. As a non-limiting example, in Table 5, Sample ID s19740672 comprising UGT ID/UGT protein m2502914 (SEQ ID NO: 111) produced: M%: 0.00%; MI%: 16.55%; MII%: 65.22%; MIII%: 10.42%; MIV%: 7.81%; and MV%: 0.00%.

TABLE 5

Secondary Glycosylation Activity by Candidate UGTs

| Sample ID | UGT ID/ UGT protein SEQ ID NO | Type | M% | MI% | MII % | MIII% | MIV% | MV% |
|---|---|---|---|---|---|---|---|---|
| s19740672 | m2502914 SEQ ID NO: 111 | Library | 0.00% | 16.55% | 65.22% | 10.42% | 7.81% | 0.00% |
| s19740675 | m2502880 SEQ ID NO: 109 | Library | 0.00% | 20.30% | 67.76% | 11.95% | 0.00% | 0.00% |
| s19740692 | m2502896 SEQ ID NO: 110 | Library | 0.00% | 16.70% | 56.01% | 9.57% | 17.72% | 0.00% |
| s19740695 | m2503038 SEQ ID NO: 113 | Library | 0.00% | 14.87% | 55.70% | 23.73% | 5.70% | 0.00% |
| s19740739 | m2502872 SEQ ID NO: 108 | Library | 0.00% | 20.49% | 63.88% | 8.82% | 5.21% | 1.60% |
| s19740756 | m2502772 SEQ ID NO: 106 | Library | 0.00% | 13.87% | 58.08% | 26.45% | 1.60% | 0.00% |
| s19740799 | m2502788 | Library | 0.00% | 6.16% | 18.74% | 8.30% | 0.00% | 66.80% |
| s19740815 | m2503014 SEQ ID NO: 112 | Library | 0.00% | 14.40% | 56.89% | 9.87% | 18.84% | 0.00% |
| s19741001 | m2503040 SEQ ID NO: 114 | Library | 0.00% | 8.29% | 18.56% | 43.77% | 28.76% | 0.61% |
| s21014696 | m69976 | Negative Control | 0.00% | 23.72% | 61.89% | 14.39% | 0.00% | 0.00% |
| s21014698 | m1385369 | Positive Control | 0.00% | 4.27% | 6.65% | 18.06% | 22.68% | 48.34% |

### Example 4: Bioinformatic Analysis of UGTs

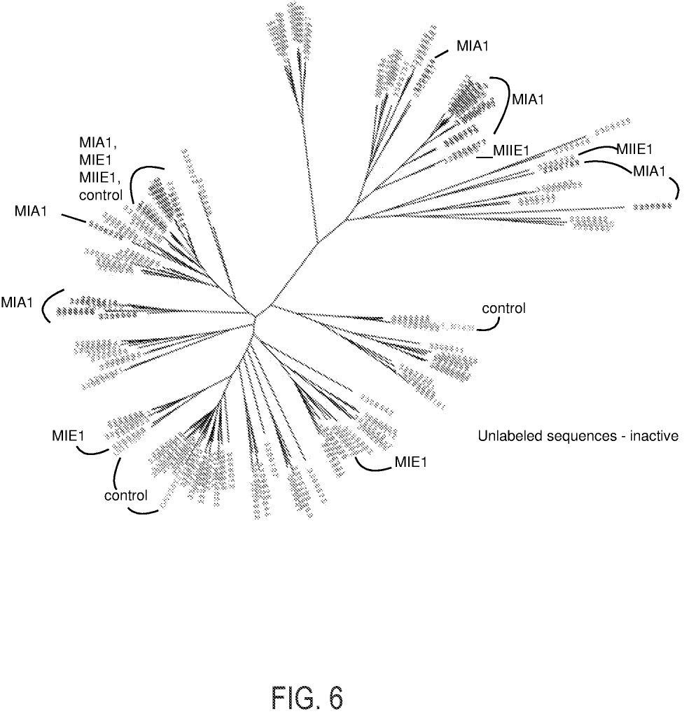

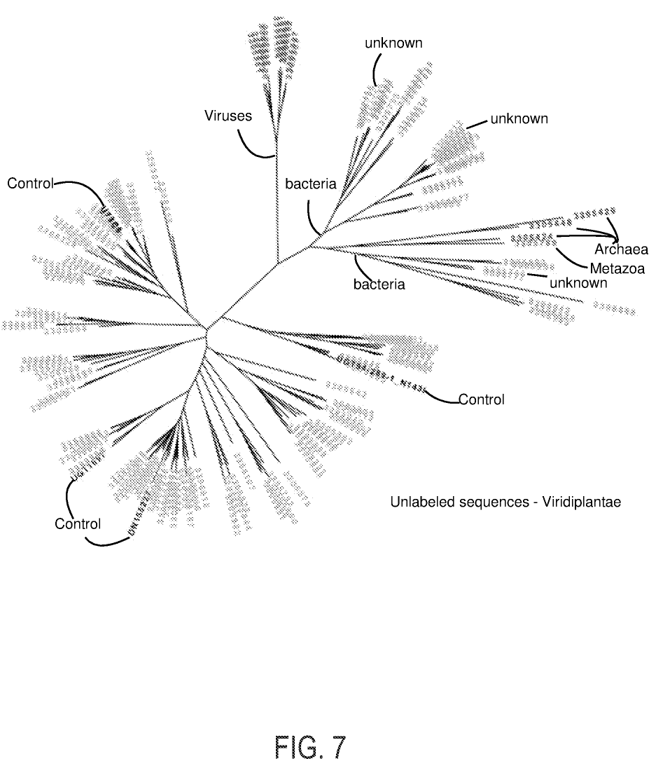

This Example describes bioinformatic analysis conducted on UGTs, including candidate UGTs identified in Examples 1-3. UGTs were clustered by MIA1, MIE1, and MIIE1 activity (FIG. 6). UGTs were also clustered by the Kingdom of Life origin classification. (FIG. 7). PhyML was used to analyze the phylogeny. FigTree was used to generate the schematic.

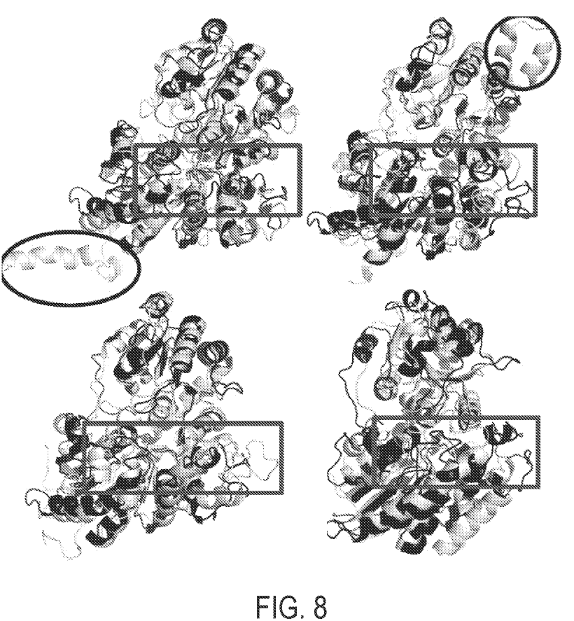

The putative structures of several UGTs that were capable of primary glycosylation were also analyzed. As shown in FIG. 8, m3710903 (SEQ ID NO: 65), m3953944 (SEQ ID NO: 75), m3710885 (SEQ ID NO: 62), and m3954606 (SEQ ID NO: 89) differed structurally relative to a reference UGT (FIG. 8). For example, rectangles highlight structural motifs (e.g., Loop 6, Alpha Helix 3, Alpha Helix 5, Loop 11, Alpha Helix 6, Loop 12 from Table 1) that differ between each candidate UGT and the reference UGT. The circles highlight sequence insertions.

### Example 5: Validation of Primary UGT Enzyme Hits From the Initial Glycosylation Activity Screen Described in Example 1

This example describes a follow up screen for the lead UGTs identified during a primary in vivo homologue screen. The purpose of this screen was to confirm whether the lead UGTs glycosylate at the C3 and C24 hydroxyl groups of mogrol to yield mogrosides with different glucose units that range from single glycosylated mogrosides up through penta-glycosylated mogrosides. This screen increased replication and additionally tested the lead enzymes identified during the initial UGT screen in the presence of secondary UGT activity. In this screen, lead primary UGT enzymes were inserted into the screening strain from Example 1, which also expressed the secondary UGT, UGT94-289-1. A total of 31 UGTs were screened in this experiment in triplicate.

To test the UGT homologues for primary glycosylation activity, an in vivo plate assay combined with analysis by LC-MS was utilized. Plasmids carrying individual UGTs were transformed and integrated into the chromosome of a S. cerevisiae chassis strain that produces a mogrol precursor. Two mogrol production strains with different titers, and parent chassis lacking any primary UGT insertions were included as negative controls. A siamenoside I production strain was included as a positive control.

Three single colonies resulting from transformation were grown as pre-cultures containing culturing media in a shaking incubator at 30° C. for 48 hours at 1,000 rpm. After 48 hours, precultures were transferred into production media and grown in a shaking incubator at 30° C. for 96 hours at 1,000 rpm. After 96 hours, cultures were extracted with an organic solvent and tested by LC-MS analysis to evaluate mogroside production.

To quantify the various mogrosides, a thermo QE-Focus ESI with a LX2 multiplexed column setup was used. A PFP column (Accucore 150 ×2.1 mm) with 12.5 mM ammonium acetate (pH 8) and acetonitrile ramp were used for separation of the individual mogroside products for the secondary 14 min/sample analysis. The QE Focus was run using full scan (range from 133.4 mz - 2000 mz) and resolution of 70,000 in negative mode.

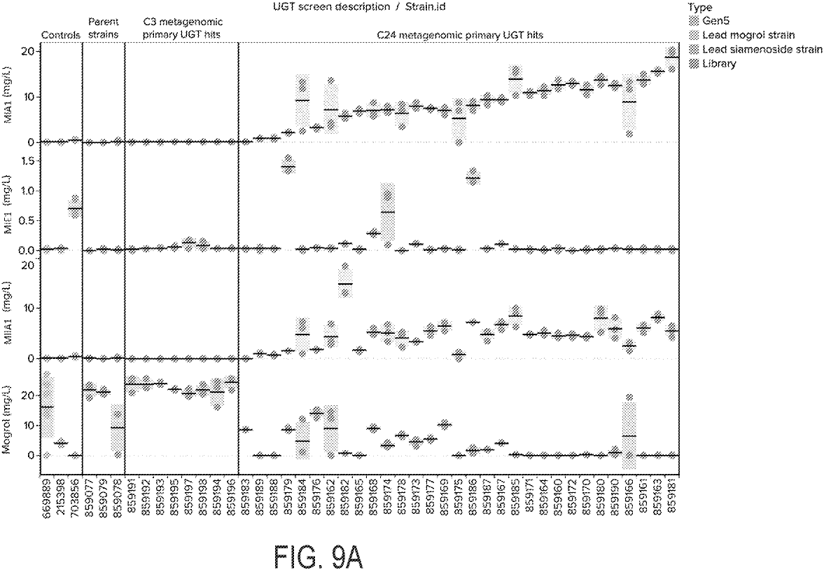

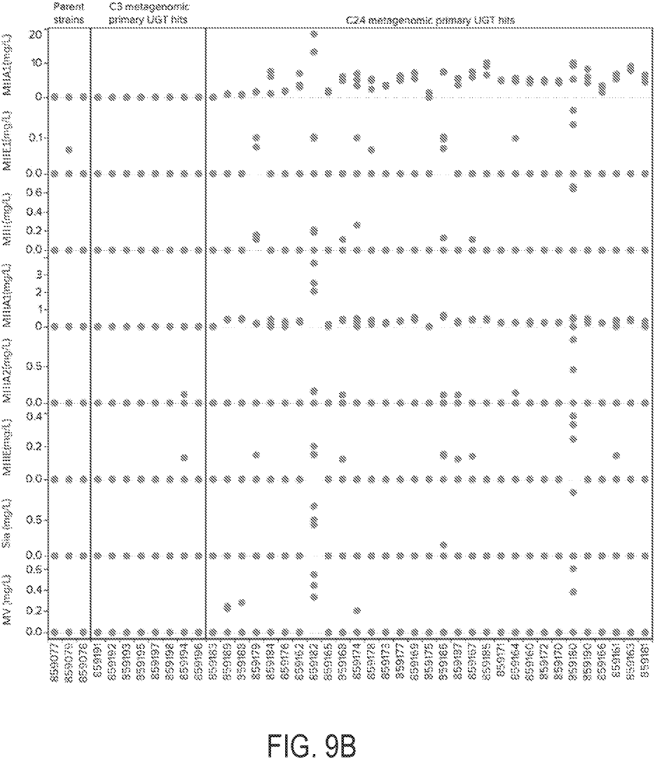

As shown in Table 6 below and in FIG. 9A, numerous UGTs showed primary UGT activity and demonstrated MIA1 production. These primary UGTs could also be used in the production of other products, including MIIA1, MIIE1, MIII, MIIIA1, MIIIA2, MIIIE, siamenoside I, and MV as shown in Table 7 and FIG. 9B. In Tables 6-7, the mogrol strain was engineered to express one or more copies of CYP1798, CYP5491, AtCPR1, CPR4497, SgCDS, EPH3, and AtEPH2, as well as to upregulate expression of ERG9 and ERG1 and downregulate expression of ERG7. The mogrol strain was used as the host cell base strain. A different UGT was added to each parent strain. One parent strain comprised SEQ ID NO: 121 and another parent strain comprised SEQ ID NO: 63.

TABLE 6

Primary glycosylation by candidate UGTs

| Strain ID | UGT ID/ UGT protein (SEQ ID NO) | MIA1 [mg/L] | MIE1 [mg/L] | MIIA1 [mg/L] | Mogrol [mg/L] |
|---|---|---|---|---|---|
| 859181 | m3955387 (SEQ ID NO: 101) | 18.71 | 0.02 | 5.50 | 0.07 |
| 859163 | m3954606 (SEQ ID NO: 89) | 15.69 | 0.02 | 8.25 | 0.10 |
| 859185 | m3953702 (SEQ ID NO: 71) | 13.93 | 0.02 | 8.50 | 0.18 |
| 859161 | m3953871 (SEQ ID NO: 73) | 13.76 | 0.02 | 6.11 | 0.00 |
| 859180 | m3953983 (SEQ ID NO: 76) | 13.75 | 0.02 | 8.11 | 0.00 |
| 859172 | m3954761 (SEQ ID NO: 97) | 12.98 | 0.00 | 4.64 | 0.00 |
| 859160 | m3954531 (SEQ ID NO: 88) | 12.60 | 0.03 | 4.52 | 0.00 |
| 859190 | m3954308 (SEQ ID NO: 85) | 12.57 | 0.04 | 6.00 | 0.84 |
| 859170 | m3954742 (SEQ ID NO: 95) | 11.63 | 0.01 | 4.40 | 0.19 |
| 859171 | m3954751 (SEQ ID NO: 96) | 10.96 | 0.02 | 4.80 | 0.00 |
| 859187 | m3954350 (SEQ ID NO: 86) | 9.40 | 0.03 | 4.74 | 1.95 |
| 859167 | m3954639 (SEQ ID NO: 92) | 9.30 | 0.11 | 6.78 | 4.13 |
| 859184 | m3953464 (SEQ ID NO: 69) | 9.19 | 0.02 | 4.81 | 4.83 |
| 859166 | m3710904 (SEQ ID NO: 66) | 8.89 | 0.02 | 2.54 | 6.51 |
| 859186 | m3954168 (SEQ ID NO: 78) | 8.11 | 1.21 | 7.24 | 1.59 |
| 859173 | m3953944 (SEQ ID NO: 75) | 7.92 | 0.11 | 3.31 | 4.43 |
| 859177 | m3954997 (SEQ ID NO: 98) | 7.39 | 0.01 | 5.51 | 5.44 |
| 859162 | m3954610 (SEQ ID NO: 90) | 7.19 | 0.03 | 4.43 | 8.99 |
| 859174 | m3953189 (SEQ ID NO: 67) | 7.09 | 0.64 | 5.12 | 3.31 |
| 859169 | m3954637 (SEQ ID NO: 91) | 6.99 | 0.03 | 6.51 | 10.13 |
| 859168 | m3954669 (SEQ ID NO: 93) | 6.96 | 0.28 | 5.26 | 9.09 |
| 859165 | m3710903 (SEQ ID NO: 65) | 6.87 | 0.02 | 1.63 | 0.08 |
| 859178 | m3955013 (SEQ ID NO: 99) | 6.33 | 0.00 | 4.03 | 6.59 |
| 859175 | m3953262 (SEQ ID NO: 68) | 5.21 | 0.01 | 0.80 | 0.00 |
| 859176 | m3953567 (SEQ ID NO: 70) | 3.18 | 0.04 | 1.76 | 14.08 |
| 859179 | m3953734 (SEQ ID NO: 72) | 2.13 | 1.40 | 1.49 | 8.67 |
| 859189 | m3954259 (SEQ ID NO: 81) | 0.86 | 0.03 | 0.90 | 0.00 |
| 859188 | m3954283 (SEQ ID NO: 83) | 0.84 | 0.03 | 0.61 | 0.00 |
| 859183 | m3954307 (SEQ ID NO: 84) | 0.06 | 0.03 | 0.00 | 8.66 |
| 859196 | m3955521 (SEQ ID NO: 103) | 0.06 | 0.04 | 0.00 | 24.36 |
| 859194 | m3953937 (SEQ ID NO: 74) | 0.06 | 0.03 | 0.00 | 21.20 |
| 859198 | m3954239 (SEQ ID NO: 79) | 0.05 | 0.08 | 0.00 | 21.82 |
| 859195 | m3955517 (SEQ ID NO: 102) | 0.05 | 0.05 | 0.00 | 22.11 |
| 859193 | m3710885 (SEQ ID NO: 62) | 0.02 | 0.03 | 0.00 | 23.98 |
| 859191 | m3954446 (SEQ ID NO: 87) | 0.02 | 0.02 | 0.00 | 23.77 |
| 859192 | m3710890 (SEQ ID NO: 63) | 0.02 | 0.03 | 0.00 | 23.75 |
| 215398 | Control Gen5 | 0.02 | 0.03 | 0.02 | 4.08 |
| 669889 | Control lead mogrol strain | 0.02 | 0.02 | 0.02 | 16.04 |
| 703856 | Control lead siamenoside I | 0.43 | 0.69 | 0.34 | 0.02 |
| 859077 | Parent strain 1 | 0.00 | 0.00 | 0.03 | 21.76 |
| 859078 | Parent strain 2 | 0.14 | 0.01 | 0.08 | 9.27 |
| 859079 | Parent strain 3 | 0.00 | 0.02 | 0.00 | 21.04 |

TABLE 7

Production of additional products by candidate UGTs

| Strain ID | UGT ID/ UGT protein (SEQ ID NO) | MIIE1 [mg/L] | MIII [mg/L] | MIIIAI [mg/L] | MIIIA2 [mg/L] | MIIIE [mg/L] | Sia [mg/L] | MV [mg/L] |
|---|---|---|---|---|---|---|---|---|
| 859181 | m3955387 (SEQ ID NO: 101) | 0.00 | 0.00 | 0.13 | 0.00 | 0.00 | 0.00 | 0.00 |
| 859163 | m3954606 (SEQ ID NO: 89) | 0.00 | 0.00 | 0.34 | 0.00 | 0.00 | 0.00 | 0.00 |
| 859185 | m3953702 (SEQ ID NO: 71) | 0.00 | 0.00 | 0.40 | 0.00 | 0.00 | 0.00 | 0.00 |
| 859161 | m3953871 (SEQ ID NO: 73) | 0.00 | 0.00 | 0.21 | 0.00 | 0.05 | 0.00 | 0.00 |
| 859180 | m3953983 (SEQ ID NO: 76) | 0.10 | 0.43 | 0.29 | 0.44 | 0.32 | 0.29 | 0.33 |
| 859172 | m3954761 (SEQ ID NO: 97) | 0.00 | 0.00 | 0.21 | 0.00 | 0.00 | 0.00 | 0.00 |
| 859160 | m3954531 (SEQ ID NO: 88) | 0.00 | 0.00 | 0.22 | 0.00 | 0.00 | 0.00 | 0.00 |
| 859190 | m3954308 (SEQ ID NO: 85) | 0.00 | 0.00 | 0.30 | 0.00 | 0.00 | 0.00 | 0.00 |
| 859170 | m3954742 (SEQ ID NO: 95) | 0.00 | 0.00 | 0.21 | 0.00 | 0.00 | 0.00 | 0.00 |
| 859171 | m3954751 (SEQ ID NO: 96) | 0.00 | 0.00 | 0.21 | 0.00 | 0.00 | 0.00 | 0.00 |
| 859187 | m3954350 (SEQ ID NO: 86) | 0.00 | 0.00 | 0.25 | 0.03 | 0.04 | 0.00 | 0.00 |
| 859167 | m3954639 (SEQ ID NO: 92) | 0.00 | 0.04 | 0.37 | 0.00 | 0.05 | 0.00 | 0.00 |
| 859184 | m3953464 (SEQ ID NO: 69) | 0.00 | 0.00 | 0.18 | 0.00 | 0.00 | 0.00 | 0.00 |
| 859166 | m3710904 (SEQ ID NO: 66) | 0.00 | 0.00 | 0.18 | 0.00 | 0.00 | 0.00 | 0.00 |
| 859186 | m3954168 (SEQ ID NO: 78) | 0.09 | 0.04 | 0.59 | 0.04 | 0.10 | 0.05 | 0.00 |
| 859173 | m3953944 (SEQ ID NO: 75) | 0.00 | 0.00 | 0.20 | 0.00 | 0.00 | 0.00 | 0.00 |
| 859177 | m3954997 (SEQ ID NO: 98) | 0.00 | 0.00 | 0.31 | 0.00 | 0.00 | 0.00 | 0.00 |
| 859162 | m3954610 (SEQ ID NO: 90) | 0.00 | 0.00 | 0.25 | 0.00 | 0.00 | 0.00 | 0.00 |
| 859174 | m3953189 (SEQ ID NO: 67) | 0.03 | 0.09 | 0.21 | 0.00 | 0.00 | 0.00 | 0.07 |
| 859169 | m3954637 (SEQ ID NO: 91) | 0.00 | 0.00 | 0.45 | 0.00 | 0.00 | 0.00 | 0.00 |
| 859168 | m3954669 (SEQ ID NO: 93) | 0.00 | 0.04 | 0.36 | 0.03 | 0.04 | 0.00 | 0.00 |
| 859165 | m3710903 (SEQ ID NO: 65) | 0.00 | 0.00 | 0.08 | 0.00 | 0.00 | 0.00 | 0.00 |
| 859178 | m3955013 (SEQ ID NO: 99) | 0.02 | 0.00 | 0.26 | 0.00 | 0.00 | 0.00 | 0.00 |
| 859175 | m3953262 (SEQ ID NO: 68) | 0.00 | 0.00 | 0.00 | 0.00 | 0.00 | 0.00 | 0.00 |
| 859176 | m3953567 (SEQ ID NO: 70) | 0.00 | 0.00 | 0.16 | 0.00 | 0.00 | 0.00 | 0.00 |
| 859179 | m3953734 (SEQ ID NO: 72) | 0.09 | 0.14 | 0.16 | 0.00 | 0.05 | 0.00 | 0.00 |
| 859189 | m3954259 (SEQ ID NO: 81) | 0.00 | 0.00 | 0.39 | 0.00 | 0.00 | 0.00 | 0.16 |
| 859188 | m3954283 (SEQ ID NO: 83) | 0.00 | 0.00 | 0.42 | 0.00 | 0.00 | 0.00 | 0.09 |
| 859183 | m3954307 (SEQ ID NO: 84) | 0.00 | 0.00 | 0.00 | 0.00 | 0.00 | 0.00 | 0.00 |
| 859196 | m3955521 (SEQ ID NO: 103) | 0.00 | 0.00 | 0.00 | 0.00 | 0.00 | 0.00 | 0.00 |
| 859194 | m3953937 (SEQ ID NO: 74) | 0.00 | 0.00 | 0.00 | 0.04 | 0.04 | 0.00 | 0.00 |
| 859198 | m3954239 (SEQ ID NO: 79) | 0.00 | 0.00 | 0.00 | 0.00 | 0.00 | 0.00 | 0.00 |
| 859195 | m3955517 (SEQ ID NO: 102) | 0.00 | 0.00 | 0.00 | 0.00 | 0.00 | 0.00 | 0.00 |
| 859193 | m3710885 (SEQ ID NO: 62) | 0.00 | 0.00 | 0.00 | 0.00 | 0.00 | 0.00 | 0.00 |
| 859191 | m3954446 (SEQ ID NO: 87) | 0.00 | 0.00 | 0.00 | 0.00 | 0.00 | 0.00 | 0.00 |
| 859192 | m3710890 (SEQ ID NO: 63) | 0.00 | 0.00 | 0.00 | 0.00 | 0.00 | 0.00 | 0.00 |
| 215398 | Control Gen5 | 0.00 | 0.00 | 0.00 | 0.00 | 0.00 | 0.00 | 0.00 |
| 669889 | Control lead mogrol strain | 0.00 | 0.00 | 0.00 | 0.00 | 0.00 | 0.00 | 0.00 |
| 703856 | Control lead siamenoside I | 0.20 | 0.80 | 0.15 | 0.29 | 0.69 | 6.15 | 7.05 |
| 859077 | Parent strain 1 | 0.00 | 0.00 | 0.00 | 0.00 | 0.00 | 0.00 | 0.00 |
| 859078 | Parent strain 2 | 0.00 | 0.00 | 0.00 | 0.00 | 0.00 | 0.00 | 0.00 |
| 859079 | Parent strain 3 | 0.02 | 0.00 | 0.00 | 0.00 | 0.00 | 0.00 | 0.00 |

### Example 6: Combining Recombinant Proteins to Produce a Mogrol Precursor, Mogrol, or a Mogroside.

The recombinant proteins of the present disclosure are used in combination to produce a mogrol precursor, (e.g., 2-3-oxidosqualene, 2,3,22,23-dioxidosqualene, cucurbitadienol, 24, 25-expoxycucurbitadienol, 24,25-dihydroxycucurbitadienol), mogrol, or mogrosides (e.g., mogroside I-A1 (MIA1), mogroside I-E (MIE), mogroside II-A1 (MIIA1), mogroside III-A1 (MIIIA1), mogroside II-E (MIIE1), mogroside III (Mill), siamenoside I, mogroside IV, mogroside III-E (MIIIE), mogroside V, and mogroside VI).

For example, to produce mogrol, genes encoding enzymes such as an SQE, a CDS, an EPH and a C11 hydroxylase are expressed in yeast cells. In some instances, a cytochrome P450 reductase is also expressed in the yeast cells. Non-limiting examples of suitable SQEs, EPHs, C11 hydroxylases and cytochrome P450 reductases are provided in Tables 10-11. Non-limiting examples of CDSs are provided in Table 9. Mogrol can be quantified using LC-MS. UGTs are further expressed in the yeast cells to produce mogrosides. Non-limiting examples of UGTs are provided in Table 8.

Yarrowia cells may be used as the yeast cell. In some instances, Yarrowia cells comprising a squalene epoxidase, a CDS, a C11 hydroxylase, a secondary UGT, and a primary UGT comprising a sequence selected from the group consisting of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 103, 74, 79, 102, 62, and 87 are used to produce one or more mogrosides. In some instances, the squalene epoxidase is encoded by the ERG1 gene. In some instances, a Yarrowia cell further comprises a cytochrome P450 reductase (CPR), a cytochrome b5, another epoxidase and/or an epoxide hydrolase. In some instances, the other epoxidase is CYP1798.

Alternatively, the recombinant proteins are purified from host cells and the mogrol is produced outside of the host cells. The recombinant proteins are added either sequentially or simultaneously to a reaction buffer comprising squalene.

TABLE 8

Sequences of Candidate UGTs Associated with the Disclosure

| Sample ID | UGT ID/ UGT Name | Nucleotide Sequence SEQ ID NO: | Amino Acid Sequence SEQ ID NO: |
|---|---|---|---|
| s23958091 | m3710885 | 1 | 62 |
| s23959819 | m3710890 | 2 | 63 |
| s23959737 | m3710903 | 4 | 65 |
| s23957806 | m3710904 | 5 | 66 |
| s23958227 | m3953189 | 6 | 67 |
| s23958297 | m3953262 | 7 | 68 |
| s23955237 | m3953464 | 8 | 69 |
| s23955329 | m3953567 | 9 | 70 |
| s23955609 | m3953702 | 10 | 71 |
| s24130828 | m3953734 | 11 | 72 |
| s24129142 | m3953871 | 12 | 73 |
| s23956161 | m3953937 | 13 | 74 |
| s24128619 | m3953944 | 14 | 75 |
| s23956230 | m3953983 | 15 | 76 |
| s24129191 | m3954005 | 16 | 77 |
| s24128597 | m3954168 | 17 | 78 |
| s24130639 | m3954239 | 18 | 79 |
| s24129279 | m3954259 | 20 | 81 |
| s23955776 | m3954283 | 22 | 83 |
| s24129435 | m3954307 | 23 | 84 |
| s23955779 | m3954308 | 24 | 85 |
| s24129351 | m3954350 | 25 | 86 |
| s23959874 | m3954446 | 26 | 87 |
| s23959878 | m3954531 | 27 | 88 |
| s24128970 | m3954606 | 28 | 89 |
| s24128899 | m3954610 | 29 | 90 |
| s23958026 | m3954637 | 30 | 91 |
| s24128774 | m3954639 | 31 | 92 |
| s24128646 | m3954669 | 32 | 93 |
| s24128689 | m3954684 | 33 | 94 |
| s23957985 | m3954742 | 34 | 95 |
| s23957988 | m3954751 | 35 | 96 |
| s24128678 | m3954761 | 36 | 97 |
| s24130921 | m3954997 | 37 | 98 |
| s23955696 | m3955013 | 38 | 99 |
| s24131011 | m3955034 | 39 | 100 |
| s23957459 | m3955387 | 40 | 101 |
| s23957941 | m3955517 | 41 | 102 |
| s24129100 | m3955521 | 42 | 103 |
| s23953261 | m3953578 | 43 | 104 |
| s23953509 | m3953963 | 44 | 105 |
| s19740756 | m2502772 | 45 | 106 |
| s19740739 | m2502872 | 47 | 108 |
| s19740675 | m2502880 | 48 | 109 |
| s19740692 | m2502896 | 49 | 110 |
| s19740672 | m2502914 | 50 | 111 |
| s19740815 | m2503014 | 51 | 112 |
| s19740695 | m2503038 | 52 | 113 |
| s19741001 | m2503040 | 53 | 114 |
| s23955762 | m3778973 | 56 | 117 |
| s23956146 | | | |
| s24129705 | | | |
| 21014696 | 69976 | 57 | |

TABLE 9

Non-Limiting Examples of CDSs

| Name | Nucleotide Sequence SEQ ID NO: | Amino Acid Sequence SEQ ID NO: |
|---|---|---|
| A0A0K9RW03_m | 184 | 224 |
| AquAgaCDS1_m | 185 | 225 |
| AquAgaCDS16 | 186 | 226 |
| AquAgaCDS6 | 187 | 227 |
| BenHisCDS2_m | 188 | 228 |
| A0A0D3QY32 | 189 | 229 |
| A0A0D3QXV2 | 190 | 230 |
| CmaCh17G013880.1 | 191 | 231 |
| A0A1S3CBF6 | 192 | 232 |
| CocGraCDS4 | 193 | 233 |
| CocGraCDS6_m | 194 | 234 |
| CSPI06G07180.1 | 195 | 235 |
| CucFoeCDS | 196 | 236 |
| CucMelMakCDSS | 197 | 237 |
| CucMetCDS | 198 | 238 |
| CucPepOviCDS1_m | 199 | 239 |
| CucPepOviCDS2 | 200 | 240 |
| CucPepOviCDS3 | 201 | 241 |
| CucPepOviCDS3_m | 202 | 242 |
| Cucsa.349060.1 | 203 | 243 |
| F6GYI4 | 204 | 244 |
| GynCarCDS1 | 205 | 245 |
| GynCarCDS4 | 206 | 246 |
| K7NBZ9 | 207 | 247 |
| LagSicCDS2_m | 208 | 248 |
| Lus10014538.g_m | 209 | 249 |
| Lus10032146.g_m | 210 | 250 |
| MomChaCDS2 | 211 | 251 |
| MomChaCDS4 | 212 | 252 |
| O23909_PEA_Y118L | 213 | 253 |
| Q6BE24 | 214 | 254 |
| SecEduCDS | 215 | 255 |
| SgCDS1 | 216 | 256 |
| SgCDS_Scer1 | 217 | 257 |
| TriKirCDS10 | 218 | 258 |
| TriKirCDS4 | 219 | 259 |
| XP_006340479.1 | 220 | 260 |
| XP_008655662.1 | 221 | 261 |
| XP_010541955.1_m | 222 | 262 |
| XP_016688836.1_m | 223 | 263 |

TABLE 10

Non-Limiting Examples of C11 Hydroxylases (P450s), Cytochrome P450 Reductases, Epoxide Hydrolases (EPHs), and Squalene Epoxidases

| Enzyme | Nucleotide Sequence SEQ ID NO: | Amino Acid Sequence SEQ ID NO: |
|---|---|---|
| C11 hydroxylase | 264 | 280 |
| C11 hydroxylase (cucurbitadienol oxidase) | 265 | 281 |
| Cytochrome P450 reductase | 266 | 282 |
| Cytochrome P450 reductase | 267 | 283 |
| Epoxide hydrolase | 268 | 284 |
| Epoxide hydrolase | 269 | 285 |
| Epoxide hydrolase (epoxide hydratase) | 270 | 286 |
| Epoxide hydrolase (epoxide hydratase) | 271 | 287 |
| Epoxide hydrolase (epoxide hydratase) | 272 | 288 |
| Epoxide hydrolase (epoxide hydratase) | 273 | 289 |
| Epoxide hydrolase (epoxide hydratase) | 274 | 290 |
| Epoxide hydrolase (epoxide hydratase) | 275 | 291 |
| Epoxide hydrolase (epoxide hydratase) | 276 | 292 |
| Squalene epoxidase | 277 | 293 |
| Squalene epoxidase | 278 | 294 |
| Squalene epoxidase | 279 | 295 |

TABLE 11

Sequences of Additional Enzymes Associated with the Disclosure

| Name | mID | Nucleotide sequence SEQ ID NO: | Amino acid sequence SEQ ID NO: |
|---|---|---|---|
| CYP1798 | m1059383 | 296 | 305 |
| AtCPR1 | m967137 | 297 | 306 |
| CPR4497 | m1740376 | 298 | 307 |
| sgCDS | m1740366 | 299 | 308 |
| EPH3 | m1612980 | 300 | 309 |
| atEPH2 | m1612970 | 301 | 310 |
| ERG9 | m1028696 | 302 | 311 |
| ERG1 | m1028695 | 303 | 312 |
| ERG7 | m3331997 | 304 | 313 |
| UGT | | 314 | 319 |
| UGT | | 315 | 320 |
| UGT | | 316 | 321 |
| UGT | | 317 | 322 |
| UGT | | 318 | 323 |

It should be appreciated that sequences disclosed in this application may or may not contain secretion signals. The sequences disclosed in this application encompass versions with or without secretion signals. It should also be understood that protein sequences disclosed in this application may be depicted with or without a start codon (M). The sequences disclosed in this application encompass versions with or without start codons. Accordingly, in some instances amino acid numbering may correspond to protein sequences containing a start codon, while in other instances, amino acid numbering may correspond to protein sequences that do not contain a start codon. It should also be understood that sequences disclosed in this application may be depicted with or without a stop codon. The sequences disclosed in this application encompass versions with or without stop codons. Aspects of the disclosure encompass host cells comprising any of the sequences described in this application and fragments thereof.

### EQUIVALENTS

Those skilled in the art will recognize, or be able to ascertain using no more than routine experimentation, many equivalents to the specific embodiments of the invention described in this application. Such equivalents are intended to be encompassed by the following claims.

All references, including patent documents, disclosed in this application are incorporated by reference in their entirety, particularly for the disclosure referenced in this application.

1. A host cell that comprises a heterologous polynucleotide encoding a UDP-glycosyltransferase (UGT), wherein the UGT comprises a sequence that is at least 90% identical to any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 94 and 100, and wherein the UGT is capable of catalyzing conversion of mogrol to MIA1.

2. The host cell of claim 1, wherein the UGT comprises a sequence that is at least 90% identical to any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, and 84.

3. The host cell of claim 1 or claim 2, wherein the UGT comprises a sequence that is at least 90% identical to SEQ ID NO: 76.

4. The host cell of claim 1 or claim 2, wherein the UGT comprises a sequence that is at least 90% identical to SEQ ID NO: 66.

5. The host cell of claim 4, wherein the UGT comprises SEQ ID NO: 88.

6. The host cell of claim 1, wherein the UGT comprises the sequence of any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 94, and 100.

7. The host cell of claim 1 or claim 2, wherein the UGT comprises the sequence of any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, and 84.

8. The host cell of claim 7, wherein the UGT comprises SEQ ID NO: 76.

9. A host cell that comprises a heterologous polynucleotide encoding a UDP-glycosyltransferase (UGT), wherein the UGT comprises a sequence that is at least 90% identical to any one of SEQ ID NOs: 103, 74, 79, 102, 62, and 87 and wherein the UGT is capable of catalyzing conversion of mogrol to MIE1.

10. The host cell of claim 9, wherein the UGT comprises the sequence of any one of SEQ ID NOs: 103, 74, 79, 102, 62, and 87.

11. A host cell that comprises a heterologous polynucleotide encoding a UDP-glycosyltransferase (UGT), wherein the UGT comprises a sequence that is at least 90% identical to any one of SEQ ID NOs: 63, 77, 84, and 94, and wherein the UGT is capable of catalyzing conversion of mogrol to MIIE1.

12. The host cell of claim 11, wherein the UGT comprises the sequence of any one of SEQ ID NOs: 63, 77, 84, and 94.

13. The host cell of any one of claims 1-12, wherein the host cell further comprises one or more heterologous polynucleotides encoding one or more of: a cucurbitadienol synthase (CDS) enzyme, a C11 hydroxylase, a cytochrome P450 reductase, an epoxide hydrolase (EPH), and squalene epoxidase (SQE).

14. The host cell of claim 13, wherein the CDS enzyme comprises a sequence that is at least 90% identical to any one of SEQ ID NOs: 226, SEQ ID NO: 235, and SEQ ID NO: 232.

15. The host cell of claim 13 or 14, wherein the C11 hydroxylase comprises the sequence of any one of SEQ ID NOs: 280-281 and 305.

16. The host cell of any one of claims 13-15, wherein the cytochrome P450 reductase comprises the sequence of any one of SEQ ID NOs: 282-283 and 306-307.

17. The host cell of any one of claims 13-16, wherein the EPH comprises the sequence of any one of SEQ ID NO: 284-292 and 309-310.

18. The host cell of any one of claims 13-17, wherein the SQE comprises the sequence of any one of SEQ ID NOs: 293-295 and 312.

19. The host cell of any one of claims 1-18, wherein the host cell further comprises a secondary UGT.

20. The host cell of claim 19, wherein the secondary UGT comprises a sequence that is at least 90% identical to any one of SEQ ID NOs: 104-106, 108-114, and 121.

21. The host cell of claim 20, wherein the secondary UGT comprises the sequence of any one of SEQ ID NOs: 104-106 and 108-114.

22. The host cell of any one of claims 1-21, wherein the host cell is a yeast cell, a plant cell, or a bacterial cell.

23. The host cell of claim 22, wherein the host cell is a yeast cell.

24. The host cell of claim 23, wherein the yeast cell is a Saccharomyces cerevisiae cell.

25. The host cell of claim 23, wherein the yeast cell is a Yarrowia cell.

26. The host cell of claim 22, wherein the host cell is a bacterial cell.

27. The host cell of claim 26, wherein the bacterial cell is an E. coli cell.

28. A method of producing a mogroside comprising culturing the host cell of any one of claims 1-27 with at least one mogroside precursor.

29. The method of claim 28, wherein the mogroside precursor is selected from mogrol, MIE1, MIA1, MIIA, MIIA1, MIIE, MIII, MIIIA1, and MIIIE.

30. The method of claim 28 or 29, wherein the mogroside that is produced is selected from MIA1, MIIA1, MIIE1, MIE1, MIII, MIIIA1, MIIIE, siamenoside I, and MV.

31. A host cell that comprises a heterologous polynucleotide encoding a UDP-glycosyltransferase (UGT), wherein the UGT comprises a sequence that is at least 90% identical to any one of SEQ ID NOs: 104-106 and 108-114, and wherein the UGT is capable of producing one or more mogrosides from a mogroside precursor.

32. The host cell of claim 31, wherein the UGT comprises the sequence of any one of SEQ ID NOs: 104-106 and 108-114.

33. The host cell of claim 31 or 32, wherein the mogroside precursor is selected from mogrol, MIE1, MIA1, MIIA, MIIA1, MIIE, MIII, MIIIA1, and MIIIE.

34. The host cell of any one of claims 31-33, wherein the mogroside that is produced is selected from MIA1, MIIA1, MIIIA1, MIIE1, MIII, siamenoside I, and MIIIE.

35. The host cell of any one of claims 31-34, wherein the host cell further comprises one or more heterologous polynucleotides encoding one or more of: a cucurbitadienol synthase (CDS) enzyme, a C11 hydroxylase, a cytochrome P450 reductase, an epoxide hydrolase (EPH), and squalene epoxidase (SQE).

36. The host cell of claim 35, wherein the CDS enzyme comprises a sequence that is at least 90% identical to any one of SEQ ID NOs: 226, SEQ ID NO: 235, and SEQ ID NO: 232.

37. The host cell of claim 35 or 36, wherein the C11 hydroxylase comprises the sequence of any one of SEQ ID NOs: 280-281 and 305.

38. The host cell of any one of claims 35-37, wherein the cytochrome P450 reductase comprises the sequence of any one of SEQ ID NOs: 282-283 and 306-307.

39. The host cell of any one of claims 35-38, wherein the EPH comprises the sequence of any one of SEQ ID NOs: 284-292 and 309-310.

40. The host cell of any one of claims 35-39, wherein the SQE comprises the sequence of any one of SEQ ID NOs: 293-295 and 312.

41. The host cell of any one of claims 31-40, wherein the host cell further comprises a primary UGT.

42. The host cell of claim 41, wherein the primary UGT comprises a sequence that is at least 90% identical to any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 103, 74, 79, 102, 62, 87.

43. The host cell of claim 42, wherein the primary UGT comprises the sequence of any one of SEQ ID NOs: 76, 101, 89, 71, 73, 97, 88, 85, 95, 96, 86, 92, 69, 66, 78, 75, 98, 90, 67, 91, 93, 65, 99, 68, 70, 72, 81, 83, 84, 103, 74, 79, 102, 62, and 87.

44. The host cell of any one of claims 31-43, wherein the host cell is a yeast cell, a plant cell, or a bacterial cell.

45. The host cell of claim 44, wherein the host cell is a yeast cell.

46. The host cell of claim 45, wherein the yeast cell is a Saccharomyces cerevisiae cell.

47. The host cell of claim 45, wherein the yeast cell is a Yarrowia cell.

48. The host cell of claim 46, wherein the host cell has reduced or eliminated expression of EXG1.

49. The host cell of claim 44, wherein the host cell is a bacterial cell.

50. The host cell of claim 49, wherein the bacterial cell is an E. coli cell.

51. A method of producing a mogroside comprising culturing the host cell of any one of claims 31-50 with at least one mogroside precursor.

52. The method of claim 51, wherein the mogroside precursor is selected from mogrol, MIE1, MIA1, MIIA, MIIA1, MIIE, MIII, MIIIA1, and MIIIE.

53. The method of claim 51 or 52, wherein the mogroside that is produced is selected from MIA1, MIIA1, MIIIA1, MIIE1, MIII, siamenoside I, and MIIIE.
